(19) United States
(12) Patent Application Publication
(10) Pub. No.: US 2005/0177228 A1
(43) Pub. Date: Aug. 11, 2005

(54) Device for changing the shape of the mitral annulus

(76) Inventors: Jan Otto Solem, Stetten (CH); Per Ola Kimblad, Lund (SE); Stevan Nielsen, Rottenburg (DE); Ib Joergenson, Haigerloch (DE); Bodo Quint, Rottenburg (DE); Gerd Seibold, Ammerbuch (DE); Randolf von Oepen, Los Altos Hills, CA (US); Suk-Woo Ha, Langwiesen (CH); Karl-Ludwig Eckert, Marthalen (CH); Stefan Schreck, Vista, CA (US); Sepehr Fariabi, Newport Coast, CA (US); Tommy Conzelmann, Rangendingen (DE)

Correspondence Address: EDWARDS LIFESCIENCES CORPORATION, ONE EDWARDS WAY, LEGAL DEPARTMENT, IRVINE, CA, 92614, US

(21) Appl. No.: 11/014,273

(22) Filed: Dec. 15, 2004

Related U.S. Application Data
(60) Provisional application No. 60/530,352, filed on Dec. 16, 2003.
(60) Provisional application No. 60/547,741, filed on Feb. 25, 2004.
(60) Provisional application No. 60/624,224, filed on Nov. 2, 2004.

(51) Int. Cl. A61F 2/24

(52) U.S. Cl. 623/2.36

(57) ABSTRACT

An elongate body including a proximal and distal anchor, and a bridge between the proximal and distal anchors. The bridge has an elongated state, having first axial length, and a shortened state, having a second axial length, wherein the second axial length is shorter than the first axial length. A resorbable thread may be woven into the bridge to hold the bridge in the elongated state and to delay the transfer of the bridge to the shortened state. In an additional embodiment, there may be one or more central anchors between the proximal and distal anchors with a bridge connecting adjacent anchors.

### CROSS-REFERENCE TO RELATED APPLICATION

This application claims priority to and the benefit of U.S. Provisional Patent Application 60/530,352 filed Dec. 16, 2003 titled Device to Change the Shape of the Mitral Valve Annulus, U.S. Provisional Patent Application 60/547,741 filed Feb. 25, 2004 titled Methods and Apparatus for Treatment of Mitral Insufficiency, and U.S. Provisional Patent Application 60/624,224 filed Nov. 2, 2004 titled Device for Changing the Shape of the Mitral Annulus, the entire content of which is expressly incorporated herein by reference.

### FIELD OF THE INVENTION

This invention relates to devices and methods for heart valve repair and, more particularly, to endovascular devices and methods for improving mitral valve function using devices inserted into he coronary sinus.

### BACKGROUND

Heart valve regurgitation, or leakage from the outflow to the inflow side of a heart valve, is a common occurrence in patients with heart failure and a source of morbidity and mortality in these patients. Usually, regurgitation will occur in the mitral valve, located between the left atrium and the left ventricle, or in the tricuspid valve, located between the right atrium and right ventricle. Mitral regurgitation in patients with heart failure is caused by changes in the geometric configurations of the left ventricle, papillary muscles and mitral annulus. Similarly, tricuspid regurgitation is caused by changes in the geometric configurations of the right ventricle, papillary muscles, and tricuspid annulus. These geometric alterations result in mitral and tricuspid leaflet tethering and incomplete coaptation in systole.

Mitral valve repair is the procedure of choice to correct mitral regurgitation of all etiologies. With the use of current surgical techniques, between 40% and 60% of regurgitant mitral valves can be repaired depending on the surgeon's experience and the anatomic conditions. The advantages of mitral valve repair over mitral valve replacement are well documented. These advantages include better preservation of cardiac function and reduced risk of anticoagulant-related hemorrhage, thromboembolism and endocarditis.

In current practice, mitral valve surgery requires an extremely invasive approach that includes a chest wall incision, cardiopulmonary bypass, cardiac and pulmonary arrest, and an incision on the heart itself to gain access to the mitral valve. Such a procedure is associated with high morbidity and mortality. Due to the risks associated with this procedure, many of the sickest patients are denied the potential benefits of surgical correction of mitral regurgitation. In addition, patients with moderate, symptomatic mitral regurgitation are denied early intervention and undergo surgical correction only after the development of cardiac dysfunction.

More particularly, current surgical practice for mitral valve repair generally requires that the posterior mitral valve annulus be reduced in radius by surgically opening the left atrium and then fixing sutures, or sutures in combination with a support ring, to the internal surface of the annulus. This structure is used to pull the annulus back into a smaller radius, thereby reducing mitral regurgitation by improving leaflet coaptation.

This method of mitral valve repair, generally termed “annuloplasty,” effectively reduces mitral regurgitation in heart failure patients. This, in turn, reduces symptoms of heart failure, improves quality of life and increases longevity. Unfortunately, however, the invasive nature of mitral valve surgery and the attendant risks render most heart failure patients poor surgical candidates. Thus, a less invasive means to increase leaflet coaptation and thereby reduce mitral regurgitation in heart failure patients would make this therapy available to a much greater percentage of patients.

Several recent developments in minimally invasive techniques for repairing the mitral valve without surgery have been introduced. Some of these techniques involve introducing systems for remodeling the mitral annulus through the coronary sinus.

The coronary sinus is a blood vessel commencing at the coronary ostium in the right atrium and passing through the atrioventricular groove in close proximity to the posterior, lateral and medial aspects of the mitral annulus. Because of its position adjacent to the mitral annulus, the coronary sinus provides an ideal conduit for positioning an endovascular prosthesis to act on the mitral annulus and therefore reshape it.

One example of a minimally invasive technique for mitral valve repair can be found in U.S. Patent Publication No. 2003/0083,538 to Adams et al. (“the '538 publication”). The '538 publication describes a balloon expandable device insertable into the coronary sinus to reshape the mitral valve annulus, the device taking the form of a frame structure having an elongated base and integral columnar structures extending therefrom. The columnar structures form the force applier to apply force to discrete portions of the wall of the coronary sinus.

Another device is described in U.S. Pat. No. 6,656,221 issued to Taylor et al. (“the '221 patent”). The '835 publication describes a substantially straight rigid elongated body including relatively flexible portions to help better distribute the stress exerted on the walls of the coronary sinus.

U.S. Patent Publication 2002/0183838 to Liddicoat et al. (“the '838 publication) describes multiple devices for minimally invasive mitral valve repair. In one embodiment, the '838 publication describes a device including an internal member having a plurality of slots and an external member having a plurality of slots. When the slots on the internal member are aligned with the slots on the external member, the device is flexible so as to follow the natural curvature of the coronary sinus. When the slots on both members are oriented away from each other, the device is straight and rigid and able to apply an anteriorly-directed force to the mitral valve annulus.

In another embodiment, the '838 publication describes an elongated body having a “w” shape. When the body is positioned in the coronary sinus, the center of the “w” is directed towards the anterior mitral annulus and inverts the natural curvature of the coronary sinus.

Another example of a minimally invasive technique for mitral valve repair can be found in U.S. Pat. No. 6,402,781 issued to Langberg et al. (“the '781 patent”). The '781 patent describes a two-dimensional prosthesis deployed into the coronary sinus via a delivery catheter. The tissue contacting surface of the prosthesis is provided with ridges, teeth or piercing structures that exert tension and enhance friction to engage to discrete portions of the wall of the coronary sinus. Moreover, the device provides an open loop through the coronary sinus and the entire coronary venous system with control lines that extend outside of the patient.

Another device is described in U.S. Pat. No. 6,790,231 to Liddicoat et al. (“the '231 patent”) . The '231 patent describes a two-dimensional elongated body having a guide wire that controls a spine of the elongated body to form an arc. The elongate body has discrete barbs along its spine to apply frictional force to discrete portions of the wall of the coronary sinus.

U.S. Pat. No. 6,676,702 to Mathis (“the '702 patent”) describes a two-dimensional mitral valve therapy device that forms an arc inside the coronary sinus to exert force on the mitral annulus. A guide wire extending from the device changes the shape of the device and the device applies pressure on discrete portions of the coronary sinus.

Despite recent attempts at minimally invasive repair of the mitral annulus using devices residing in the coronary sinus, there is a need for such endovascular correction devices that do not require an external member, such as a wire, to alter the shape of the device, yet still provide enough force to reshape the mitral annulus. Further, there is a need for devices, including those that use an external member, that are less traumatic to the sinus, both during and after their insertion into the coronary sinus, and are also more reliable over long periods of time. Finally, there is a need for better control over the shape in which the mitral annulus is deformed by such endovascular correction devices.

### SUMMARY

The invention described herein provides a more reliable and a safer way to treat a dilated mitral annulus. Devices in accordance with principles of the present invention may comprise one or more components suitable for deployment in the coronary sinus and adjoining coronary veins. The devices may be configured to bend in-situ to apply a compressive load to the mitral valve annulus with or without a length change, or may include multiple components that are drawn or contracted towards one another to remodel the mitral valve annulus. Any of a number of types of anchors may be used to engage the surrounding vein and tissue, including anchors comprising ultraviolet (UV) curable materials, hydrogels, hydrophilic materials, or biologically anchored components. Remodeling of the mitral valve annulus may be accomplished during initial deployment of the device, or by biological actuation during subsequent in-dwelling of the device.

One embodiment of the invention comprises an elongate body having a proximal, central and distal stent section, wherein a backbone fixes the stent sections relative to one another and wherein the central stent section has a plurality of rings connected to the backbone. The elongate body has two states: a first state wherein the elongate body has a shape that is adaptable to the shape of the coronary sinus and a second state wherein the elongate body pushes on the coronary sinus to reduce dilatation. Further, the elongate body has a greater axial length in the first state than in the second state.

When the body is deployed, the proximal and distal stent sections are expanded to act as anchors in the coronary sinus. Expansion of the central stent section foreshortens the elongate body, drawing the proximal and distal stent sections toward the central stent section, and cinching the mitral valve and closing the gap between mitral valve leaflets. When the gap between the mitral valve leaflets is closed, the effects of mitral valve regurgitation are drastically reduced or eliminated.

In another embodiment, the device comprises proximal and distal transitional sections in addition to the proximal, central and distal stent sections. The transitional sections allow the body to have enough flexibility to conform to the curvature of the coronary sinus.

Yet another embodiment comprises a proximal stent module and a distal stent module, wherein each stent module has an anchor section, a central section and a backbone. When both stent modules are inserted into the coronary sinus, the central sections of the two modules may overlap, effectively providing for one continuous stent. Additionally, based on the degree of rigidity desired, the backbones of the stents may be misaligned to provide for increased flexibility.

Yet another embodiment comprises a tubular elongate body having such dimensions so as to be insertable into the coronary sinus. The body has two states: a first state wherein the body has a linear shape adaptable to the shape of the coronary sinus and a second state, to which the body is transferable from the first state, wherein the device has a nonlinear shape.

In yet another embodiment, the invention comprises a proximal stent section, a central stent section, and a distal stent section, where a diameter of the elongate body varies from the proximal stent section to the distal stent section. The body expands into a three-dimensional shape that conforms to the anatomy of the coronary sinus, thereby applying more uniform stress to the walls of the inner radius of the coronary sinus. The device achieves remodeling of the mitral annulus through foreshortening, which reduces the overall length of the coronary sinus and as a result, reduces the circumference of the mitral annulus.

In accordance with the invention, in one embodiment, the elongate body is a multi-filament woven structure, where an angle of weave in the woven structure determines the degree of expansion force and foreshortening of the coronary sinus. The woven structure is made of metal with memory effect, such as Nitinol, Elgiloy, or spring steel.

Also in accordance with this aspect of the invention, in one embodiment a rigid inner elongated body is placed inside of the elongate body. In one example, the rigid inner elongate body is placed along the central stent section of the elongate body and fitted into the central stent section of the elongate body. The inner elongate body is made from rigid metal, such as stainless steel. Moreover, the elongate body may be self expandable or balloon expandable.

In yet another embodiment, the invention comprises a proximal and distal anchor, and a bridge between the proximal and distal anchors. The bridge has an elongated state, having first axial length, and a shortened state, having a second axial length, wherein the second axial length is shorter than the first axial length. A resorbable thread may be woven into the bridge to hold the bridge in the elongated state and to delay the transfer of the bridge to the shortened state. In an additional embodiment, there may be one or more central anchors between the proximal and distal anchors with a bridge connecting adjacent anchors.

In another embodiment of the present invention, the device comprises proximal and distal anchor elements, wherein the proximal anchor element comprises a deployable flange. The proximal and distal anchor elements are delivered into the coronary sinus in a contracted state, and then are deployed preferably within the coronary sinus so that the flange of the proximal anchor element engages the coronary sinus ostium. A cinch mechanism, for example, comprising a plurality of wires and eyelets, is provided to reduce the distance between proximal and distal anchor elements, thereby reducing the circumference of the mitral valve annulus.

To reduce trauma to the intima of the coronary sinus during actuation of the cinch mechanism, the distal anchor element preferably is chemically or mechanically bonded to the intima of the coronary sinus prior to actuation of the cinch mechanism. The distal anchor element may comprise a UV-curable material that causes the distal anchor element to bond with the intima of the coronary sinus when a UV source is provided. Alternatively, the distal anchor element may comprise a hydrogel or hydrophilic foam that causes the distal anchor element to chemically bond with the intima of the coronary sinus, which in effect may reduce trauma to the intima of the vessel wall during actuation of the cinch mechanism.

In another embodiment of the present invention, a proximal balloon catheter is used in conjunction with a distal balloon catheter to treat mitral insufficiency. The balloons of the proximal and distal catheters may be deployed spaced apart a selected distance, preferably substantially within the coronary sinus, and then manipulated so that they remodel the curvature of the coronary sinus. This remodeling in turn applies a compressive force upon the mitral valve to remodel the mitral valve annulus. With the compressive force applied, a substance, such as a biological hardening agent, may be introduced into a cavity formed between the two balloons to cause a hardened mass to form in the cavity. When the balloons of the proximal and distal catheters subsequently are removed, the mass ensures that the coronary sinus is retained in the remodeled shape.

In yet a further embodiment of the present invention, a stent is provided having proximal and distal sections coupled to one another by a central section, so that expansion and/or curvature of the central section causes the proximal and distal sections to be drawn together. In this embodiment, the central section includes one or more biodegradable structures, such as biodegradable sutures, that retain the central section in its contracted state until the vessel endothelium has overgrown a portion of the proximal and distal sections. This provides biological anchoring of the proximal and distal sections of the stent within at least a portion of the coronary sinus.

After the proximal and distal sections have become endothelialized, the biodegradable structure degrades, releasing the central section and enabling it to expand and/or assume a desired curvature. The expansion and/or curvature of the central section causes the stent to reduce the radius of curvature of the coronary sinus, thereby causing remodeling of the mitral valve annulus.

In another embodiment, a device for the treatment of mitral annulus dilatation includes a cylindrical proximal stent module having an anchor section and a central section and a cylindrical distal stent module having an anchor section and a central section, wherein the proximal and distal stent modules have two states, a first state wherein the proximal and distal stent modules have a shape that is adaptable to the shape of the coronary sinus, and a second state wherein the elongate body pushes on the coronary sinus to reduce dilatation, wherein each stent module has a backbone, and each backbone fixes the anchor section relative to the central section on each module along one side of the module, and wherein, when the proximal and distal stent modules are in the second state, the central section of the proximal stent overlaps the central section of the distal stent.

In this embodiment, the device may be inserted into a coronary sinus, and the anchor sections of the proximal stent module and the distal stent module anchor each module, respectively, to the coronary sinus when the modules are in the second state. The proximal and distal stent modules may be made from stainless steel.

In this embodiment, the stent modules may be inserted into the coronary sinus, and the backbone of the proximal stent section may be separated from the backbone of the distal stent section.

For example, the backbone of the proximal stent section may be angularly separated from the backbone of the distal stent section by between about 60°-180°.

In this embodiment, the proximal and distal stent sections may be transferable from the first state to the second state by a balloon. The proximal and distal stent modules may have a greater axial length in the first state than in the second state.

In another embodiment, a device for the treatment of mitral annulus dilatation includes a tubular elongate body having such dimensions as to be insertable into a coronary sinus, wherein the elongate body has two states, a first state wherein the elongate body has a linear shape that is adaptable to the shape of the coronary sinus, and a second state, to which the elongate body is transferable from the first state, wherein the device has a nonlinear shape.

In another embodiment, the tubular elongate body in the second state has a substantially w-shaped configuration. The elongate body may be transferable from a first state to a second state by a balloon. The elongate body may also include at least two spines. In another embodiment, the tubular elongate body further includes a plurality of interconnecting members extending between the at least two spines.

In another embodiment, a device for treatment of mitral annulus dilation includes an outer elongate body having such dimensions as to be insertable into a coronary sinus, the outer elongate body comprising a proximal stent section, a central stent section, and a distal stent section, wherein a diameter of the outer elongate body varies from the proximal stent section to the distal stent section, the outer elongate body having two states, a first state wherein the outer elongate body is adaptable to be inserted into the coronary sinus, and a second state wherein the outer elongate body expands inside the coronary sinus to provide foreshortening of the coronary sinus; and a rigid inner elongate body being placed inside of the outer elongate body when the outer elongate body is in the second state.

In another embodiment, a method of treating mitral annulus dilation includes providing an elongate body for treatment of mitral annulus dilation, the elongate body comprising a curved configuration to conform to an anatomy of a coronary sinus, the elongate body having a proximal stent section, a central stent section, and a distal stent section, wherein a diameter of the elongate body varies from the proximal stent section to the distal stent section; inserting the elongate body into the coronary sinus; expanding the elongate body into a three-dimensional shape to make substantial contact with walls of the coronary sinus; and foreshortening the elongate body.

In another embodiment, the method includes inserting a rigid inner elongate body inside the expanded elongate body using a balloon; and expanding the inner elongate body to make a substantial contact with the outer elongate body.

In another embodiment, an apparatus for treating mitral annulus dilatation includes (a) a proximal anchor element; (b) a distal anchor element adapted to be at least partially bonded to an intima of a patient's vessel; and (c) means for drawing the distal anchor element towards the proximal anchor element.

In another embodiment, the proximal anchor element further comprises a flange configured to abut a coronary ostium.

In another embodiment, the proximal anchor element comprises a self-deploying stent.

In another embodiment, the distal anchor element comprises a self-deploying stent configured to engage an intima of a patient's vessel in an expanded state.

In another embodiment, the distal anchor element further comprises an expandable foam member having proximal and distal ends and a bore extending therebetween, wherein the foam member is configured to engage an intima of a patient's vessel in an expanded state.

In another embodiment, the foam member comprises a hydrophilic foam.

In another embodiment, the distal anchor element further comprises a light-reactive binding agent.

In another embodiment, a catheter having proximal and distal ends, a lumen extending therebetween, and at least one port disposed at the distal end, wherein the catheter is configured to transmit light from the proximal end to the port via the lumen.

In another embodiment, at least one radiopaque marker band disposed on the distal end of the catheter.

In another embodiment, the distal anchor element further comprises a hydrogel.

In another embodiment, a method for treating mitral annulus dilatation includes (a) providing apparatus comprising a proximal anchor element and a distal anchor element in contracted states, (b) deploying the distal anchor element at a first location in a patient's vessel; (c) deploying the proximal anchor element at a second location in a patient's vessel; (d) bonding at least a portion of the distal anchor element to an intima of the patient's vessel; and (e) drawing the distal anchor towards the proximal anchor element to apply a compressive force upon the mitral annulus.

In another embodiment, the distal anchor element is chemically bonded to an intima of a patient's coronary sinus.

In another embodiment, the method further includes (a) providing a light-reactive binding agent disposed on at least a portion of the distal anchor element; (b) providing a light source; and (c) exposing the light-reactive binding agent to the light source to cause at least a portion of the distal anchor element to polymerize.

In another embodiment, the method further includes (a) providing a hydrogel disposed on at least a portion of the distal anchor element; and (b) causing the hydrogel to harden.

In another embodiment, the method further includes (a) providing a hydrophilic foam member; and (b) causing the hydrophilic foam member to engage an intima of the patient's coronary sinus and or great cardiac vein.

In another embodiment, a method for treating mitral annulus dilatation includes (a) providing a first balloon catheter having proximal and distal ends, a lumen extending therebetween, and a balloon disposed at the distal end; (b) providing a second balloon catheter having proximal and distal ends, a lumen extending therebetween, and a balloon disposed at the distal end; (c) deploying the balloon of the first catheter at a first location in a patient's coronary sinus; (d) deploying the balloon of the second catheter at a second location in a patient's vessel, the second location being proximal to the first location; (e) drawing the balloon of the first catheter towards the balloon of the second catheter to apply a compressive force upon the mitral annulus; (f) forming a coherent mass in a cavity formed between the balloon of the first catheter and the balloon of the second catheter; (g) contracting the balloon of the first catheter and the balloon of the second catheter; and (h) removing the first catheter and the second catheter.

In another embodiment, forming a coherent mass comprises injecting a substance into the cavity.

In another embodiment, injecting the substance into the cavity comprises injecting the substance into the cavity via an annulus formed between an outer surface of the first catheter and an interior surface of the second catheter.

In another embodiment, drawing the balloon of the first catheter towards the balloon of the second catheter further comprises causing a plurality of ribs or bumps disposed about the balloon of the first catheter to engage a portion of a vessel wall.

In another embodiment, at least an exterior surface of the first catheter is coated with a non-stick adherent.

In another embodiment, an apparatus for treating mitral annulus dilatation includes (a) a stent having proximal and distal sections, wherein the proximal and distal sections have a radially contracted state suitable for insertion into a vessel and radially expanded state in which they are substantially flush with a vessel wall; and (b) a central section disposed between the proximal and distal sections, wherein the central section has a elongated state suitable for insertion into a vessel and a foreshortened state having a curvature configured to apply a compressive force to and a foreshortening force on the mitral valve annulus.

In another embodiment, one or more biodegradable structures are disposed on the central section in the contracted state.

In another embodiment, the proximal section is configured to become biologically anchored to a vessel before the one or more biodegradable structures degrade.

In another embodiment, the distal section is configured to become biologically anchored to a vessel before the one or more biodegradable structures degrade.

In another embodiment, the central section comprises a shape memory material.

In another embodiment, an apparatus for treating mitral annulus dilatation includes a stent having proximal and distal sections, wherein the proximal and distal sections have a radially contracted state suitable for insertion into a vessel and radially expanded state in which they have a diameter greater than the diameter of the vessel wall; and a central section disposed between the proximal and distal sections, wherein the central section has an elongated long state suitable for insertion into a vessel and a foreshortened state having a curvature configured to apply a compressive force upon the mitral annulus and a foreshortening force on the mitral valve annulus.

### BRIEF DESCRIPTION OF THE DRAWINGS

In the drawings, like reference characters generally refer to the same parts throughout the different views. Also, the drawings are not necessarily to scale, emphasis generally being placed upon illustrating the principles of the invention.

FIG. 1 is a three-dimensional view of the mitral valve, coronary sinus and adjacent aortic valve.

FIG. 2 is a side view of an embodiment of an elongate body of the present invention including a central stent section with a backbone and a severed region.

FIG. 3 is a perspective schematic view of the body of FIG. 2 in an expanded state.

FIG. 4 is a cross-sectional view of a mitral valve and a coronary sinus into which an embodiment of a body of the present invention and a first balloon have been inserted.

FIG. 5 is a cross-sectional view of a mitral valve and a coronary sinus in which proximal and distal sections of an embodiment of a body of the present invention have been expanded and wherein a balloon has been inserted into a central section of the body.

FIG. 6 is a side view of an embodiment of an elongate body of the present invention including a proximal and a distal transitional section.

FIG. 7 is a side view of a distal stent module of an embodiment of the present invention.

FIG. 8 is a side view of a proximal stent module of an embodiment of the present invention.

FIG. 9 is a side view of a distal and proximal stent module as they may be oriented when inserted into a coronary sinus.

FIG. 10 is a flat view of a camel stent of the present invention.

FIG. 11 is a top view of a camel stent embodiment of the present invention.

FIG. 12 is a side view of a camel stent embodiment of the present invention.

FIG. 13 is a three-dimensional view of an exemplary embodiment of an elongate body of the present invention.

FIG. 14 is another three-dimensional view of the elongate body of FIG. 13 depicted from a different angle.

FIGS. 15A-15S are side views of further alternative devices of the present invention.

FIG. 16 is a perspective view of an alternate device of the present invention.

FIG. 17 schematically depicts a first state of the elongate body of FIG. 13.

FIG. 18 schematically depicts a second state of the elongate body of FIG. 13.

FIG. 19 schematically depicts a second state of an alternate embodiment of the present invention having an outer elongate body and an inner elongate body positioned inside the coronary sinus.

FIG. 20 is a side view of an embodiment of an elongate body of the present invention including a proximal anchor, a distal anchor and a bridge having resorbable thread connecting the proximal and distal anchors.

FIG. 21 is a detail of the bridge of FIG. 20.

FIG. 22 is a side view of an embodiment of an elongate body of the present invention including a proximal anchor, a distal anchor and a central anchor with a bridge having resorbable thread connecting the anchors together.

FIG. 23 is a side view of an embodiment of an elongate body of the present invention including a proximal anchor, a distal anchor and two central anchors with a bridge having resorbable thread connecting the anchors together.

FIGS. 24A-24D describe a further embodiment of the present invention.

FIGS. 25A-25C illustrate exemplary embodiments of the anchor elements of FIGS. 24A-24D.

FIGS. 26A-26B illustrate deployment and actuation of the device of FIGS. 24A-24D.

FIGS. 27A-27L illustrate alternative embodiments of the present invention.

### DETAILED DESCRIPTION

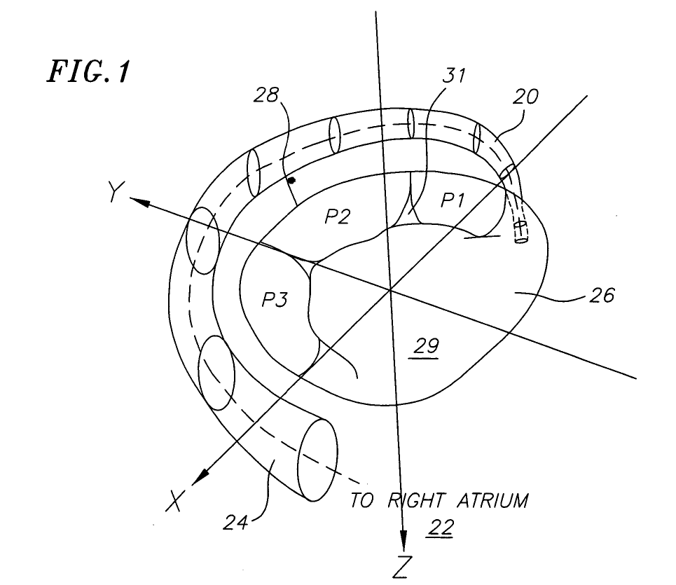

Referring to FIG. 1, a coronary sinus 20 extends from a right atrium 22 and a coronary ostium 24 and wraps around a mitral valve 26. The term coronary sinus is used herein as a generic term to describe a portion of the vena return system that is situated adjacent to the mitral valve 26 along the atrioventricular groove. The term coronary sinus 20 used herein generally includes the coronary sinus, the great cardiac vein and the anterior intraventricular vein. A mitral annulus 28 is a portion of tissue surrounding a mitral valve orifice to which several leaflets attach. The mitral valve 26 has two leaflets, an anterior leaflet 29 and a posterior leaflet 31 having three scallops P1, P2 and P3.

The problem of mitral regurgitation often results when a posterior aspect of the mitral annulus 28 dilates and displaces one or more of the posterior leaflet scallops P1, P2 or P3 away from the anterior leaflet 29. To reduce or eliminate mitral regurgitation, therefore, it is desirable to move the posterior aspect of the mitral annulus 28 in an anterior direction. For instance, in the specific case of ischemic mitral regurgitation, the posterior section of the mitral valve may dilate symmetrically or asymmetrically. In the case of symmetric dilatation, the dilation is usually more pronounced in the P2 scallop of the posterior section, while in the case of asymmetric dilatation, the dilation is usually more pronounced in the P3 scallop of the posterior section. Consequently, it is desirable to move the area of the mitral annulus 28 adjacent to the area of dilatation of the mitral valve 26 while leaving the remaining section of the mitral annulus unaltered. The catheter-based devices of the present invention can be inserted within the coronary sinus 20 to the proper location so as to perform the desired reshaping procedure on the mitral annulus 28.

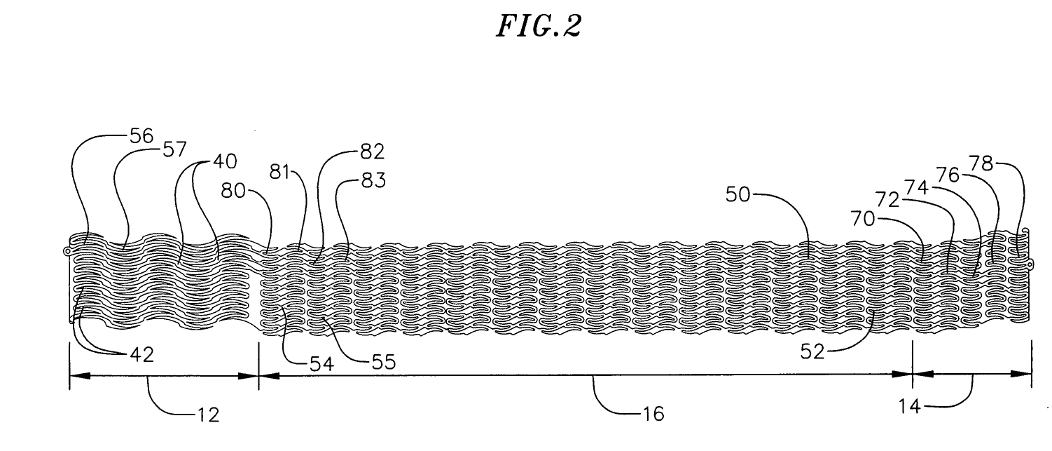

The following embodiment comprises an elongate body 10, as shown, for example, in FIG. 2. The elongate body 10 is manufactured by programming a desired pattern into a computer and cutting the pattern into a tube of stainless steel. The tube may be, however, cut by any other appropriate means. FIG. 2 is a “flat pattern” view showing the elongate body 10 cut along its axial length and laid flat.

As shown in FIG. 2, the elongate body 10 has a proximal stent section 12, a distal stent section 14, and a central stent section 16. As used herein, “distal” means the direction of the device as it is being inserted into a patient's body or a point of reference closer to the leading end of the device as it is inserted into a patient's body. Similarly, as used herein “proximal” means the direction of the device as it is being removed from a patient's body or a point of reference closer to a trailing end of a device as it is inserted into a patient's body.

The distal and proximal stent sections 14, 12 are used to anchor the body 10 into the distal and proximal ends, respectively, of the coronary sinus 20. The proximal end of the coronary sinus is located at or near the coronary sinus ostium 24. The central stent section 16 is attached between a distal end of the proximal stent section 12 and a proximal end of the distal stent section 14 and serves to “foreshorten” the coronary sinus 20. The reduction in length of a stent section when it is expanded is referred to as foreshortening.

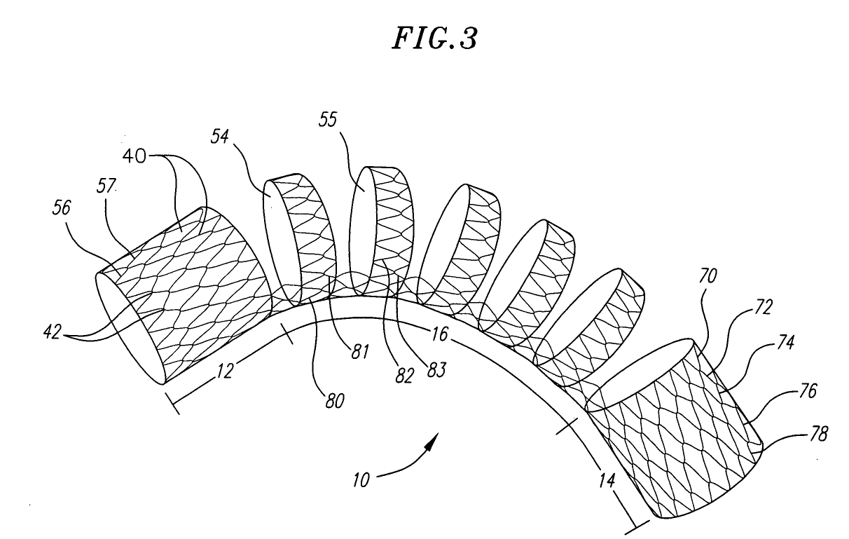

The elongate body 10 has two states, a compressed state (not shown) and an expanded state, as shown in FIG. 3. In the compressed state, the elongate body 10 has a diameter that is less than the diameter of the coronary sinus 20 and the elongate body is generally flexible enough to conform to the shape of the coronary sinus. In this state, the elongate body 10 has a substantially uniform diameter of between about 1.5 to 4 mm. In the expanded state, the elongate body 10 has a diameter that is about equal to or greater than a diameter of a non-expanded coronary sinus 20. Specifically, in the expanded state the diameter of the distal stent section 14 is between about 3 to 6 mm, the diameter of the proximal stent section 12 is between about 10 to 15 mm, and the diameter of the central stent section 16 is between about 6 to 10 mm.

Referring to FIGS. 2 and 3, one embodiment of the device comprises a tubular elongate body 10 made of stainless steel in a mesh configuration. The mesh configuration includes a series of connected stainless steel loops, for example, 56, 57. In the depicted embodiment, the loops have a zigzag shape including alternating peaks 42.

In the depicted embodiment, the proximal stent section 12 includes five loops. When a first loop 56 loop is connected to an adjacent loop 57 at at least two peaks 42, a four-sided opening 40 is formed. In an exemplary embodiment, the four-sided openings 40 of the proximal stent section have a compressed length of about 2 to 10 mm and a height of essentially 0 to 1 mm.

As shown in FIG. 2, the distal stent section 14 includes five loops. A first loop 70 and an adjacent second loop 72 are connected at each peak 42 to form a ring of four-sided openings 40. The second loop 72 is partially connected to a third loop 74 at four peaks 42 and the third loop is partially connected to a fourth loop 76 at four peaks. The fourth loop 76 is partially connected to a fifth loop 78 at two peaks. The number of loops and the number of peaks by which each loop is connected to an adjacent loop is not critical and numerous permutations are possible. However, the distal stent 14 should be flexible enough to make the body 10 steerable through the coronary sinus 20. In an exemplary embodiment, the four-sided openings 40 of the distal stent section 14 have a compressed length of about 2 to 10 mm and a height of essentially 0 to 1 mm.

As further shown in FIG. 2, the central stent section 16 separates the proximal stent section 12 and the distal stent section 14. The connections between the stent sections 12, 14 and 16 are flexible joints to allow the stent to conform to the local curvature of the coronary sinus 20. For example, in the depicted embodiment, the central stent section 16 is partially connected to the proximal stent section 12 at three peaks 42 and it is also connected to the distal stent section 14 at three peaks.

The central stent section 16 includes twenty-eight loops. In this section, a first loop 80 is joined to a second loop 81 at every peak to form a first ring 54. Further, a third loop 82 is joined to a fourth loop 83 to form a second ring 55. The adjacent first and second rings 54, 55 are partially connected to each other at three peaks 42. The central stent section 16 of the depicted embodiment includes fourteen rings each partially connected to an adjacent ring at three peaks. The structure of the rings allows the axis of the central stent section 16 to conform to the curvature of the coronary sinus 20. The region of the central stent section 16 that forms continuous four-sided openings 40, i.e. where the peaks 42 of adjacent rings are connected to each other, is a backbone 50. The region of the central stent section 16 where the rings are not connected to each other is a severed region 52. In an exemplary embodiment, the four-sided openings 40 of the central stent section 16 have a compressed length of about 2 to 10 mm and a height of essentially 0 to 1 mm. Again, the number of loops and the number of peaks by which each loop is connected to an adjacent loop is not critical and numerous permutations are possible.

The device of the first embodiment is deployed as follows. As shown in FIG. 4, the elongate body 10, in the compressed state, is mounted onto a first balloon 58, which acts as a delivery catheter. The first balloon 58 has a length generally corresponding tn the length of the distal stent section 14 and is inserted so that it is enveloped by the distal stent section. The elongate body 10 and the first balloon 58 are inserted into the coronary sinus 20 from the coronary sinus ostium 24, e.g., until the central stent section 16 is generally aligned with the P2 scallop. Once the elongate body 10 and the first balloon 58 are positioned in the coronary sinus, the first balloon is expanded by introducing, for example, a saline solution through the delivery catheter and into the balloon. Alternately, any biocompatible solution may be used to inflate the balloon. The force of the expansion of the first balloon 58 expands the distal stent section 14 so that its circumference is forced against the circumference of the coronary sinus 20 and anchors it into the wall of the coronary sinus. Once the distal stent section 14 is anchored, the first balloon 58 is deflated and removed.

A second balloon (not shown) having a length generally corresponding to the length of the proximal stent section 12 is then inserted into the elongate body 10 so that it is enveloped by the proximal stent section. The second balloon is then expanded as above using a saline solution to fill the balloon. The expansion force of the second balloon expands the proximal stent section 12 so that its circumference is forced against the coronary sinus 20 and anchors it to the wall of the coronary sinus. The second balloon is then deflated and removed. In one embodiment, the proximal stent section 12 is sized such that expansion of the proximal stent section makes it into a funnel shape adjacent to the right atrium 22. The funnel shape conforms to the coronary sinus ostium 24 to help secure the proximal stent section 12 in place.

Although the described method of deployment and expansion of the stent sections involves expanding the distal section prior to expanding the proximal section, it will be appreciated that the proximal section may be expanded prior to the distal section. In addition, the same balloon or different balloons, or balloons shorter or longer than the proximal and distal stent sections may be used as desired.

Once both the proximal and distal stent sections 12, 14 have been expanded and anchored to the coronary sinus 20, a third balloon 62 is inserted into the elongate body 10 so that it is enveloped by the central stent section 16 as shown in FIG. 5. The third balloon 62 has a length generally corresponding to the length of the central stent section 16. The central stent section 16 is then expanded by filling the third balloon 62 with a saline solution. The severed regions 52 of the central stent section 16 allow the body 10 the flexibility to generally conform to the shape of the coronary sinus 20 as the body expands.

In an alternate embodiment, a shorter balloon may be used to expand the central stent section 16 in sections to achieve the desired diameters along the central stent section. By expanding the central stent section 16 in sections, the amount of foreshortening of the coronary sinus 20 can be more accurately adjusted.

When the central stent section 16 expands, the length of the four-sided openings 40 is reduced as the height of the four-sided openings is increased. The body 10 is designed such that when it is expanded, it has a curved shape that generally follows the anatomical curvature of the coronary sinus 20. Additionally, as a result of the reduction in the length of the four-sided openings 40, the length of the entire central stent section 16 is foreshortened. The foreshortening of the central stent section 16 pulls the distal stent section 14 and the proximal stent section 12 toward each other. As a result, the distance between the proximal and distal stent sections 12, 14 is reduced. Since the proximal and distal stent sections 12, 14 are anchored to the walls of the coronary sinus 20, the length of the coronary sinus is thereby also reduced. The reduction in length of the coronary sinus 20 cinches the coronary sinus more tightly around the P1, P2 and P3 scallops of the mitral valve 26 and pushes one or more of the scallops, closer to the anterior leaflet 29 of the mitral valve. This allows a gap between the anterior leaflet 29 and the P1, P2 and P3 scallops of the posterior leaflet 31 to close. When the gap between the mitral valve leaflets is closed, the effects of mitral valve regurgitation are drastically reduced or eliminated.

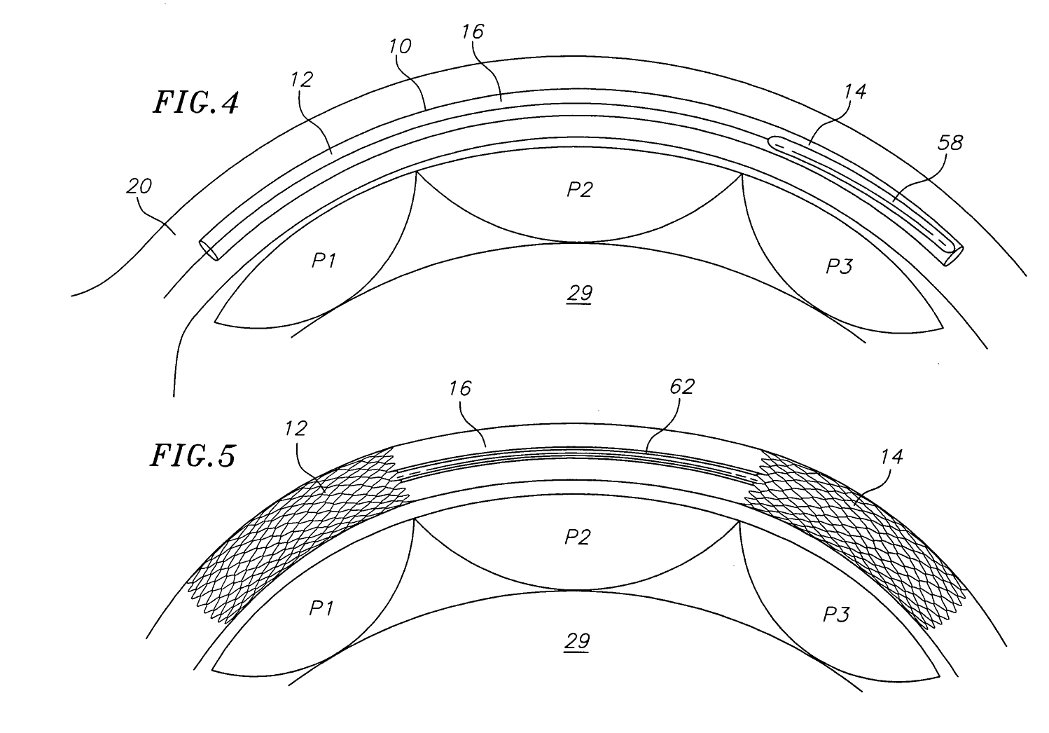

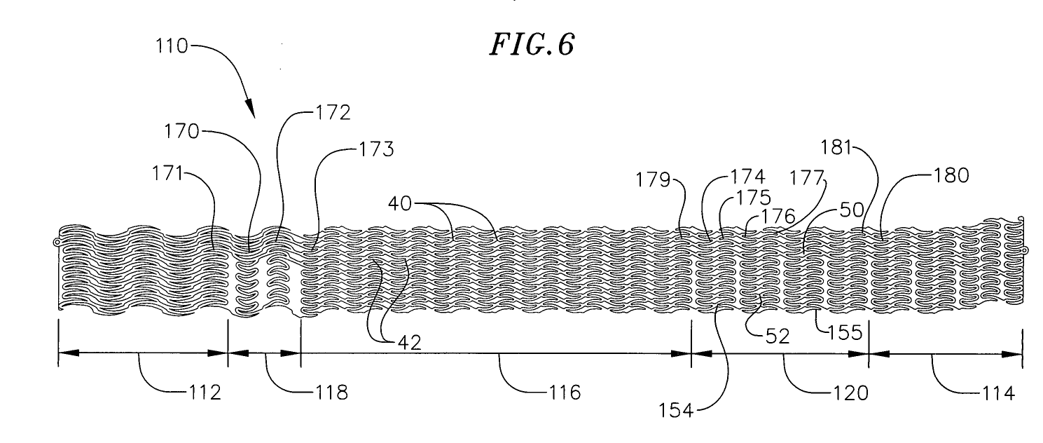

A second embodiment of the elongate body is shown in FIG. 6. In this embodiment, an elongate body 110 has a mesh configuration similar to that described with respect to the previous embodiment. In addition to a distal stent section 114, a proximal stent section 112, and a central stent section 116, the second embodiment also includes a distal transitional section 120 and a proximal transitional section 118. The distal and proximal stent sections 114, 112 are used to anchor the body 110 into the distal and proximal ends, respectively, of the coronary sinus 20. The distal and proximal transitional sections 120, 118, located between the central stent section 116 and the distal and proximal stent sections 114, 112, respectively, provide a flexible transition zone for improved load distribution. In addition, the transitional sections 112 and 120 may experience significant foreshortening during expansion providing the additional benefit of coronary sinus contraction.

The second embodiment is similar to the first embodiment in that it has two states, a compressed state and an expanded state. Further, the structure of the proximal and distal stent sections 112, 114 are identical to those of the first embodiment. The purpose of these flexible stent sections 112 and 114 is to provide a large conforming contact area between the stent and the outer wall of the coronary sinus 20 which better distributes the force exerted on the body 110 by the vessel wall. The central stent section 116 includes eighteen loops to form seventeen rings of four-sided openings 40. Since each ring of the central stent section 116 of the second embodiment is connected to the ring adjacent to it at each peak 42, the rings form a continuous mesh configuration.

The proximal transitional section 118 of the second embodiment is connected to the distal end of the proximal stent section 112 and the proximal end of the central stent section 116. The proximal transitional section 118 includes two loops. As shown in FIG. 6, a first loop 170 is connected to a most distal loop 171 of the proximal stent section 112 at three peaks 42 and a second loop 172 is connected to a most proximal loop 173 of the central stent section 116 at three peaks. The first loop 170 is also connected to the second loop 172 at three peaks 42 along the same axis as it is connected to the proximal and central stent sections 112, 116, thus forming a backbone 50 and a severed region 52 for flexibility similar to the central stent section 116 of the first embodiment. It will be appreciated that a fewer number or greater number of loops may be used in the proximal transitional section 118, or no loops, wherein the proximal stent section 112 is connected to the central stent section 116.

As also shown in FIG. 6, the distal transitional section 120 is located between a distal end of the central stent section 116 and a proximal end of the distal stent section 114. Specifically, a most proximal loop 174 in the distal transitional section 120 is partially connected to a distal-most loop 179 in the central stent section 116 at three peaks and a distal-most loop 181 in the distal transitional section 120 is partially connected to a proximal-most loop 180 in the distal stent section at three peaks. The distal transitional region 120 includes ten loops. The first loop 174 in the distal transitional section 120 is joined to a second loop 175 at every peak to form a first ring 154. Further, a third loop 176 is joined to a fourth loop 177 to form a second ring 155. The adjacent rings 154 and 155 are partially connected to each other at three peaks 42. The distal transitional section 120 of the present embodiment includes five such rings each connected to an adjacent ring at three peaks. The region that forms continuous four-sided openings 40 is a backbone 50 and the region where the rings are not connected is a severed region 52. It will be appreciated that a fewer number or greater number of loops may be used in the distal transitional section 120, or no loops, wherein the distal stent section 114 is connected to the central stent section 116.

The proximal and distal stent sections 112 and 114 of the second embodiment are deployed as described above with respect to the first embodiment. The elongate body 110 is positioned in the coronary sinus 20 so that the central stent section 116 is generally aligned with the P2 scallop of the posterior leaflet 31 of the mitral valve 26. In an alternate embodiment, the distal stent section 114 may be of increased flexibility to allow for placement in the proximal region of the great cardiac vein (not shown). In addition, the same balloon or different balloons, or balloons shorter or longer than the proximal and distal stent sections may be used as desired.

Once both the proximal and distal stent sections 112, 114 are balloon expanded and anchored to the coronary sinus 20, a third balloon (not shown) having a length generally corresponding to the combined lengths of the central stent section 116, the proximal transitional stent section 118 and the distal transitional stent section 120 is inserted into the elongate body 110 so that it is enveloped by all three stent sections 116, 118 and 120. These three sections 116, 118, 120 are then expanded using the third balloon. As the central stent section 116 is expanded, its rigidity straightens a central section of the coronary sinus. As the coronary sinus 20 straightens, the P1, P2 and/or P3 scallops, of the mitral valve 26 are moved anteriorly, thereby closing the gap between the scallops and the anterior leaflet 29 of the mitral valve 26. Additionally, expanding the central stent section 116 and the proximal and distal transitional sections 118, 120 foreshortens the elongate body 110, reducing the distance between the proximal and distal stent sections 112, 114 and cinching the coronary sinus 20 more tightly around the P1, P2 and P3 scallops. The severed region 52 of the transitional sections 118, 120 allows the elongate body 110 the flexibility to generally conform to the curvature of the coronary sinus 20 as the body expands.

Alternatively, a shorter balloon may be used to expand the central stent section 116, proximal transitional section 118 and distal transitional section 120 in steps to achieve the desired diameters along the central stent section 116. By expanding the central stent section 116 in parts, the amount of foreshortening and straightening of the coronary sinus 20 can be better adjusted.

Inserting a stent deep into the coronary sinus 20 toward the anterior intraventricular vein may sometimes be difficult because of the curved shape of the distal region of the coronary sinus. Therefore, the distal part of a device insertable into the coronary sinus 20 needs to be flexible. One possible way to achieve a more flexible stent is to reduce the wall thickness of a stent and provide for a more flexible design of the stent. On the other hand, using two overlapping stents allows for a flexible stent in the curvy distal region of the coronary sinus 20 and a stronger, more rigid part in the proximal region. More specifically, the area where two stents overlap will have a higher radial strength and become more rigid when it is expanded. This rigidity in turn will provide a more effective straightening effect in the desired area of the coronary sinus 20.

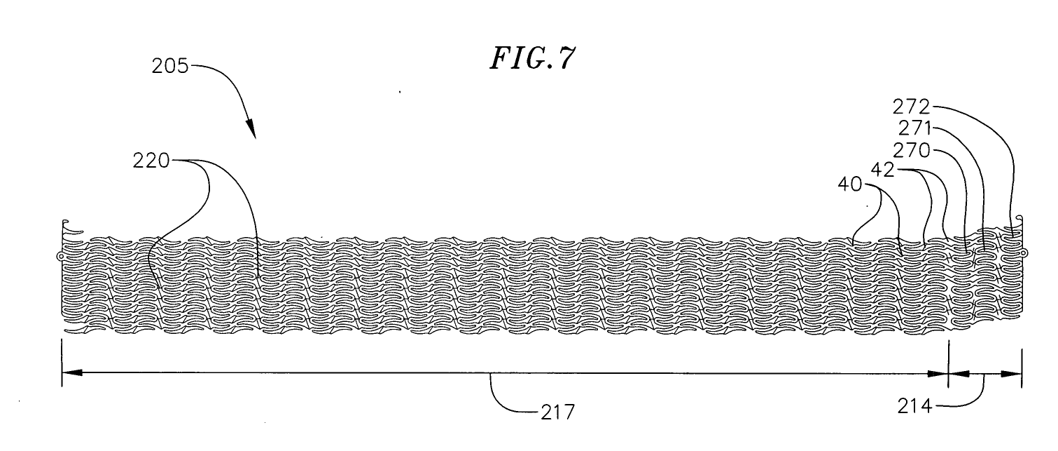

In that regard, a third embodiment of the present invention, as shown in FIGS. 7 and 8, comprises a proximal stent module 200 (FIG. 8) and a distal stent module 205 (FIG. 7). Both the proximal and distal stent modules 200, 205 have a compressed and expanded state, as described above with respect to the previous embodiments.

In one embodiment, the distal stent module 205 has an anchor section 214, located at the distal end of the distal stent module, and a central section 217. The anchor section 214 includes three loops. A first loop 270 is connected to a second loop 271 at four peaks 42 and the second loop is connected to a third loop 272 at two peaks. Accordingly, the distal stent module will be more flexible in the distal direction. The central stent section 217 includes thirty-six loops. As with respect to the first embodiment described above, alternating pairs of loops are connected at each peak to form rings of four-sided openings 40. Each ring is connected to an adjacent ring at three peaks, where the connected portion forms a backbone 250 and the unconnected portion forms a severed region similar to the central stent section 16 of the first embodiment. FIGS. 7 and 8 both include lines 220 in places of the modules 200 and 205 where larger pieces of material will be removed by laser cutting. These single lines 220 represent a cut to be made by the laser that will allow the large pieces of material to be more easily removed while leaving the remaining material undamaged.

As shown in FIG. 8, the proximal stent module 200 has an anchor section 212, located at the proximal end of the proximal stent module 200, and a central section 215. The anchor section 212 is a combination of the proximal stent section 112 and the proximal transitional section 118 as described above with respect to the second embodiment. The central section 215 includes twenty-four loops. Similarly to the central section 217 of the distal stent module 205, alternating pairs of loops are connected at each peak to form rings of four-sided openings 40. Each ring is connected to an adjacent ring at three peaks 42, where the connected portion forms a backbone 254 and the unconnected portion forms a severed region.

The device of the third embodiment is deployed as follows. The distal stent module 205 in a compressed state is mounted onto a first balloon (not shown), which acts as a delivery catheter. The first balloon has a length generally corresponding to the length of the anchor section 214 and is inserted so that it is enveloped by the anchor section. The distal stent module 205 and the first balloon are inserted into the coronary sinus 20 from the coronary sinus ostium 24 so that the central section 215 is generally aligned with, e.g., the P2 scallop. Once the distal stent module 205 and the first balloon are positioned in the coronary sinus 20, the first balloon is expanded by introducing a saline solution through the delivery catheter and into the balloon. The balloon expands the distal stent module 205 so that the module's circumference is forced against to the circumference of the coronary sinus 20 and so that the module is anchored to the wall of the coronary sinus. Once the distal stent module 205 is anchored, the first balloon is deflated and removed.

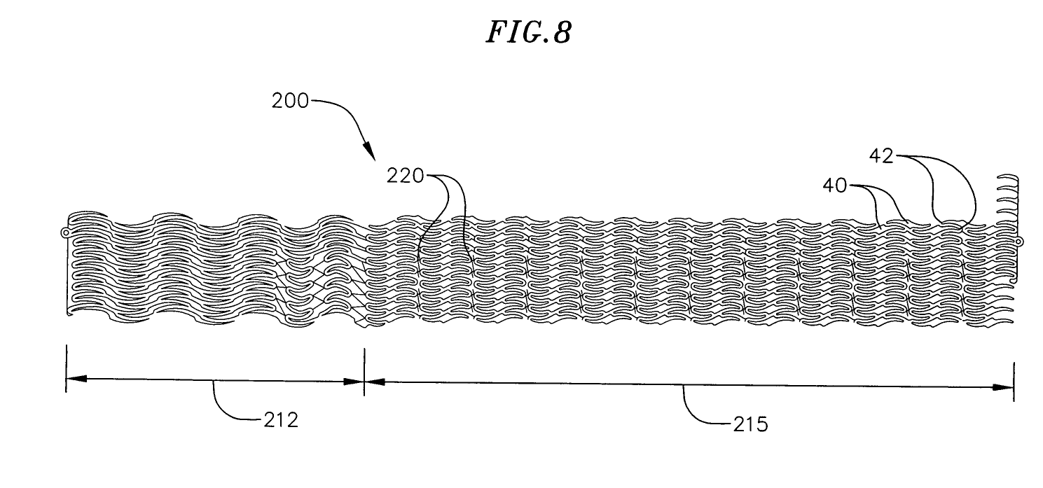

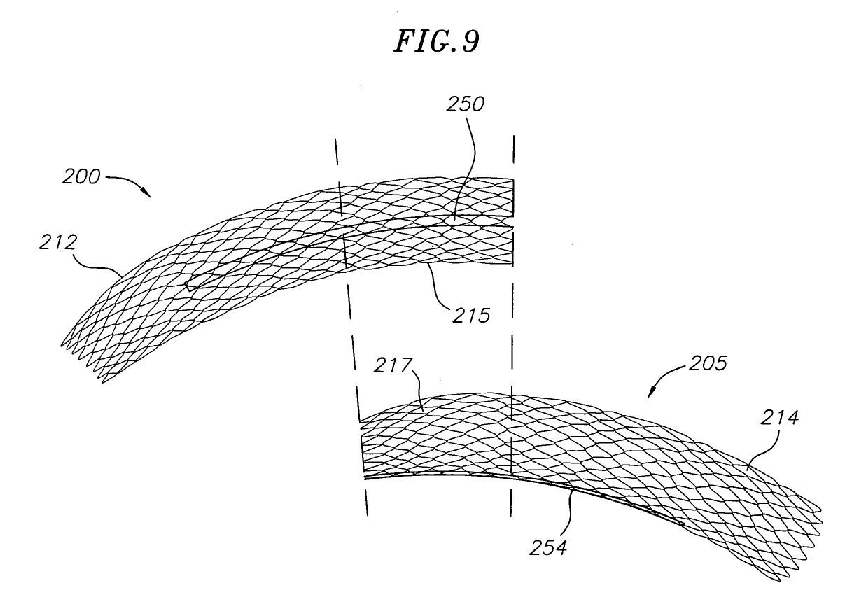

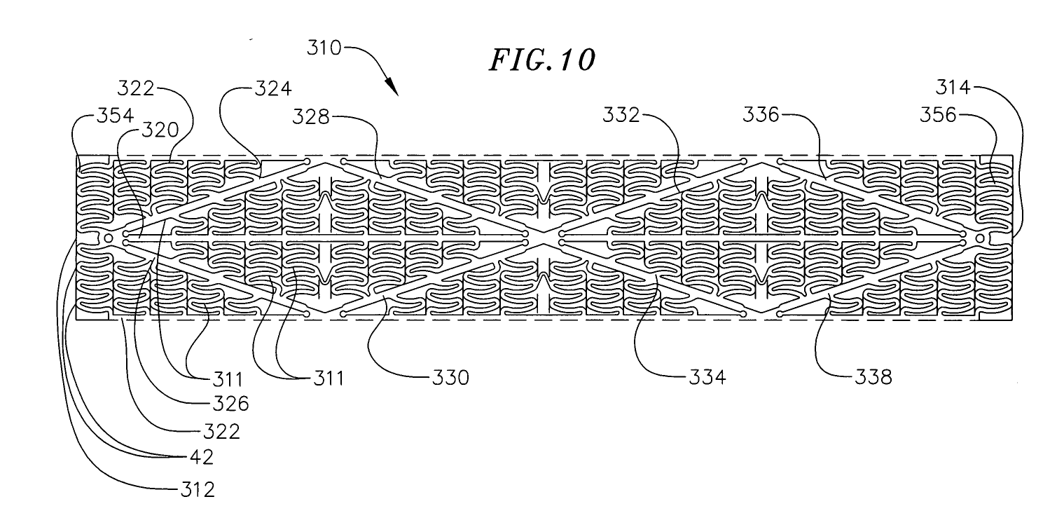

A second balloon (not shown) is then mounted on the proximal stent module 200, the second balloon having a length corresponding to the length of the anchor section 212. The proximal stent module 200 and the second balloon are then inserted into the coronary sinus so that the central section 215 of the proximal stent module 200 overlaps the central section 217 of the distal stent module 205 by at least about 2 cm. Further, as shown in FIG. 9, upon insertion, the backbone 250 of the proximal stent module 200 is angularly separated from the backbone 254 of the distal stent module 205 depending on the anatomy of the patient and the desired rigidity of the overlapping section. Although the backbones 250 and 254 may be aligned, in alternate embodiments the backbones are separated by about 60°-180°. The closer the backbones 250, 254 are together, the less rigid the overlapping section will be. On the other hand, if the backbones 250 and 254 are spaced 180° apart, the overlapping section will be as rigid as possible and able to provide the most strength to straighten the coronary sinus 20.

Once the proximal stent module 200 is in place, the second balloon 260 is expanded using a saline solution to fill the balloon. The balloon expands the proximal stent module 200 so that the module's circumference is forced against the circumference of the coronary sinus 20 and so that the module is anchored to the wall of the coronary sinus. Once the proximal stent module 200 is anchored, the second balloon is deflated and removed. In addition, the same balloon or different balloons, or balloons shorter or longer than the proximal and distal stent sections may be used as desired.

Once the proximal and distal stent modules 200, 205 have been anchored in the coronary sinus, a third balloon (not shown) is inserted. The third balloon has a length generally corresponding to the entire length of the combined central sections 215 and 217, i.e., the balloon extends the entire distance between the anchor sections 212 and 214. The third balloon is then expanded using a saline solution, and such expansion simultaneously expands the central sections 215 and 217 so that these sections have a circumferences of approximately the circumference of the coronary sinus 20. The proximal and distal stent modules 200, 205 effectively become one stent as they expand due to the overlapping region of the central stent sections 215 and 217 becoming secured together as a result of the proximal stent module 200 expanding into the distal stent module 205. The expanded central sections 215, 217 serve to straighten the coronary sinus 20 and push the posterior leaflet 31 of the mitral valve 26 anteriorly. Further, expanding the central sections 215 and 217 foreshortens the “combined” stent and cinches the coronary sinus around the P1, P2 and/or P3 scallops, of the posterior leaflet 31.

A fourth embodiment of the invention comprises a “camel” stent 310. The camel stent is an elongate tubular member having two diametrically opposed spines 320 and 322. FIG. 9 is a “flat pattern” view showing the camel stent 310 cut along its axial length and laid flat. In this case, the stent 310 has been cut along one spine 322 of the two spines 320, 322 running the length of the stent. In an exemplary embodiment, the length of the stent 310 is about 40 to 120 mm. The stent 310 includes two stainless steel loops 354 and 356, each loop having a zigzag shape with alternating peaks 42. One loop 354 is located at a proximal end 312 and one loop 356 is located at a distal end 314 of the stent 310. Extending between the loops 354 and 356 are the two spines 320 and 322 spaced 1802 apart. In a proximal half of the stent 310, angularly extending about one quarter the length of the stent from the first spine 320 to the second spine 322 are first and second interconnecting members 324, 326. At the location where the first two interconnecting members 324, 326 meet the second spine 322, a third and a fourth interconnecting member 328, 330 extend angularly about one quarter of the length of the stent 310 from the second spine 322 to the first spine 324. The third and fourth interconnecting members 328, 330 meet the first longitudinal member 320 at about the middle of the camel stent 310. The distal half of the stent 310 is a mirror image of the proximal half, the distal half having two interconnecting members 332, 334 that extend from the first spine 320 to the second spine 322 and two interconnecting members 336, 338 extend from the second spine 322 to the first spine 320.

On the proximal half of the stent extending between the first and second interconnecting members 324, 326 bisected by the second spine 322 are four strands 311 of zigzag shaped stainless steel having at least one peak 42. Similarly, there are four strands 311 extending between the first and second interconnecting members 324, 326 bisected by the first spine 320. Further, four strands 311 extend between the third and fourth interconnecting members 328, 330 and are bisected by the second spine 322 and four strands are bisected by the first spine 320. The structure of the distal half of the stent 310 is a mirror image of the structure of the proximal half of the stent.

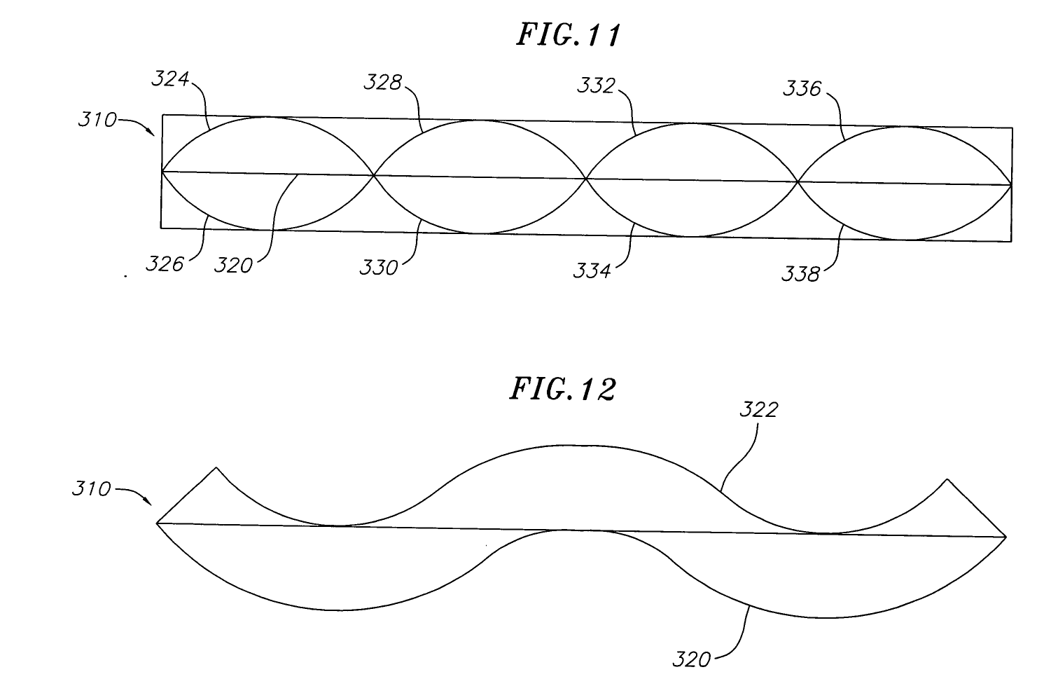

The camel stent 310 has two states, a compressed state and an expanded state. In the compressed state, the camel stent 310 has a diameter that is less that the diameter of the coronary sinus 20 and the stent is flexible enough to be suitably located in the coronary sinus. In this state, the camel stent 310 has a substantially uniform diameter of about 1.5 to 4 mm. In the expanded state, as shown in FIGS. 11 and 12 the camel stent is generally “w” shaped and has a diameter of about 4 to 12 mm.

The camel stent 310 is deployed as follows. The camel stent is mounted on a balloon catheter (not shown). The balloon has a length generally corresponding to the entire length of the camel stent 310. The camel stent 310 and the balloon are inserted into the coronary sinus 20 from the coronary sinus ostium 24 so that the center of the stent is generally aligned, e.g., with the P2 scallop. Once the stent 310 is positioned in the coronary sinus 20, the balloon is expanded using a saline solution, as described above. The expansion of the zigzag shaped strands 311 and the structure of the spines 320, 322 and interconnecting members 324, 326, 328, 330, 332, 334, 336 and 338 causes the expanded stent 310 to have a substantially w-shaped structure.

The “w” shape of the camel stent 310 in its expanded state anchors the camel stent inside the coronary sinus 20. Further, since the center of the stent 310 is adjacent to the P2 scallop, it pushes the P2 scallop anteriorly, thereby closing the gap between the anterior leaflet 29 and posterior leaflet 31 of the coronary sinus 20. In other embodiments, the design of the camel stent 310 may be modified to have only a single bend, two bends or more than three bends and/or may have a nonuniform diameter. Additionally, the camel stent 310 may be part of a stent system having proximal and distal stent sections.

FIG. 13 shows yet another embodiment of the invention comprising an elongate body 1300. In this embodiment, the elongate body 1300 self expands into a three-dimensional shape that conforms to the anatomy of the coronary sinus, thereby applying substantially uniform stress to the walls of the coronary sinus 20. Such expansion of the elongate body 1300 achieves remodeling of the mitral annulus through foreshortening, which reduces the overall length of the coronary sinus 20 and, in turn, reduces the circumference of the mitral annulus 28.

As illustrated in FIG. 1, the coronary sinus 20 is a curved tubular structure that enwraps the posterior leaflet 31 of the mitral valve 26 with scallops P1, P2, and P3. The coronary sinus 20, as shown, has a central portion Y located in an x-y plane defining the annulus of the mitral valve 26. A proximal portion of the coronary sinus 20 extends slightly upwardly out of the x-y plane towards the coronary ostium 24 of the right atrium 22. A distal portion X of the coronary sinus 20 extends downwardly behind the P1 scallop out of the x-y plane into the great cardiac vein and anterior interventricular vein.

The diameter of the coronary sinus 20 decreases from the proximal end to the distal end of the coronary sinus 20. The diameter of the central section of the coronary sinus 20 remains generally uniform throughout its length.

FIG. 13 illustrates a three-dimensional view of an embodiment of the elongate body 1300 in its unstressed, natural state. The elongate body 1300 is compressible to permit insertion into the coronary sinus 20 percutaneously and has the ability to self expand into a three-dimensional shape to conform to the anatomy of the coronary sinus 20. The elongate body 1300 has a proximal stent section 1305, a central stent section 1310, and a distal stent section 1315, each of which conforms generally in size and shape to the part of the coronary sinus 20 into which it will be inserted. In one exemplary embodiment, in its unstressed state, the diameter of the elongate body 1300 along its length is greater than the diameter of the coronary sinus 20 along its length for reasons to be discussed below. The proximal and distal stent sections 1305 and 1315 are used to anchor the elongate body 200 into the proximal and distal ends, respectively, of the coronary sinus 20. The central stent section 1310 is attached between a distal end of the proximal stent section 1305 and a proximal end of the distal stent section 1315. After the elongate body is deployed in the coronary sinus, the central stent section 1310 is located in the x-y plane shown in FIG. 13 generally aligned, for example, with the P2 scallop along the posterior leaflet 31 of the mitral valve 26 (FIG. . The proximal stent section 1305 extends slightly upwardly out of the x-y plane towards the coronary ostium 24. The distal stent section 1315 extends downwardly behind the P1 scallop extending out of the x-y plane into the great cardiac vein.

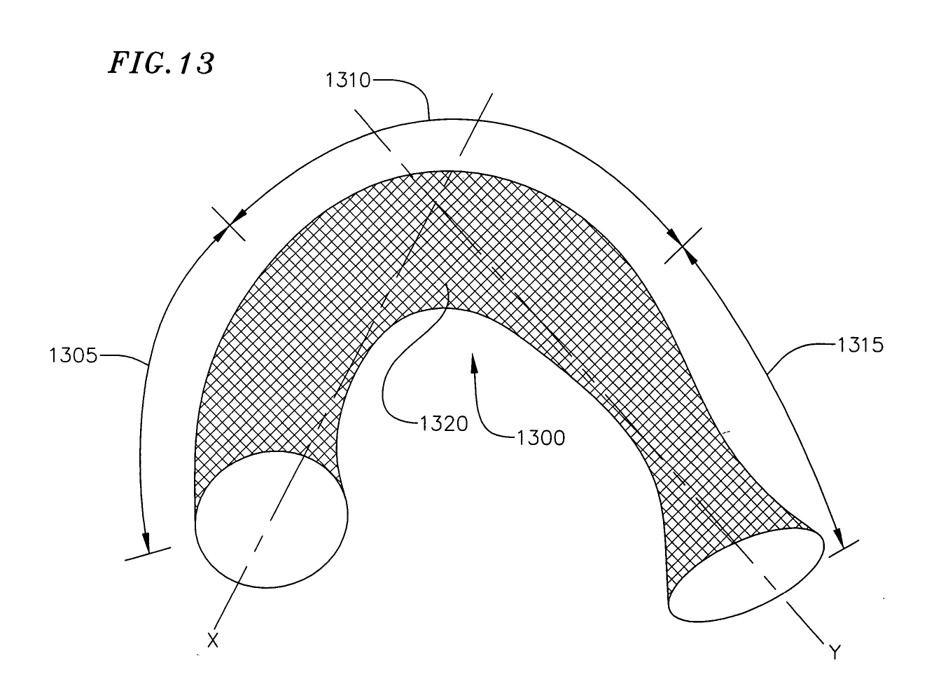

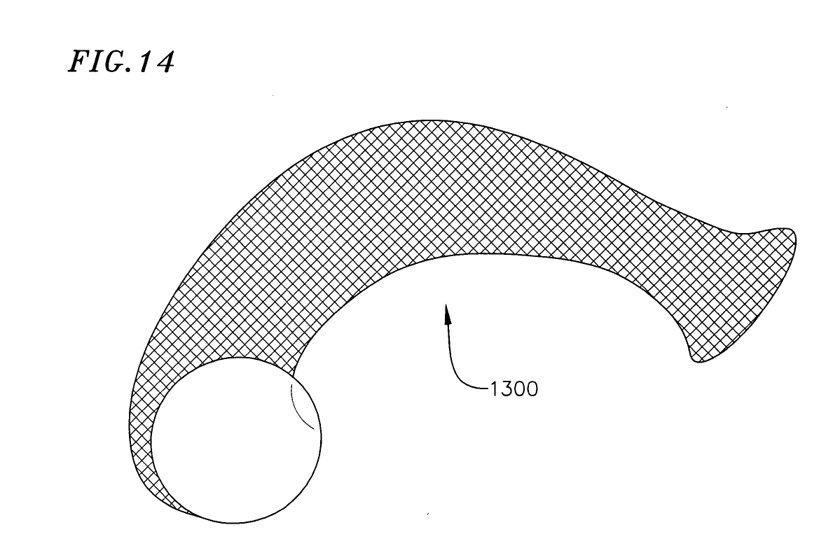

FIG. 14 illustrates another three-dimensional view of the embodiment of the elongate body 1300 depicted from a different angle wherein the viewer is looking into the proximal end of the elongate body. As shown in FIG. 14, to better emulate the slight upward extension of the proximal portion of the coronary sinus 20, the end of the proximal stent section 1305 slightly bends and faces upward. Moreover, the slightly upward facing end of the proximal stent section 1305 and the downward facing end of the distal stent section 1315 of the elongate body 1300 flare out in a funnel shape to securely anchor the elongate body to the wall of the coronary sinus 20.

To match with the varying diameters of the coronary sinus 20, the diameter of the elongate body 1300 decreases from the proximal stent section 1305 to the distal stent section 1315 and the diameter of the central stent section 1310 remains generally uniform. In one embodiment, for the elongate body 1300 having the initial total length of about 155 mm, the proximal stent section 1305 has the diameter of about 22 mm, the central stent section 1310 has the diameter of about 6 mm, the distal stent section 1315 has the diameter of about 11 mm in its unstressed state. In another embodiment of the elongate body 1300 also having the initial total length of about 155 mm, the proximal stent section 1305 has the diameter of about 21 mm, the central stent section 1310 has the diameter of about 8 mm and the distal stent section 1315 has the diameter of about 19 mm in its unstressed state.

Furthermore, referring again to FIG. 13, to conform with a radial arc of the coronary sinus along the x-y plane of the P2 scallop, a radial arc 1320 of the central stent section 1310 of the elongate body 1300 arches along the x-y plane in the range of 90 to 150 degrees in its unstressed state.

Referring again to FIG. 13, the elongate body 1300 has a multi-filament woven structure made from shape metal with memory effect, such as, but not limited to, Nitinol, Elgiloy, or spring steel. The self-expansion force and the anchoring force of the elongate body 1300, which affects the degree of foreshortening of the coronary sinus 20, is controlled by various factors, such as the angle of the weave (i.e., intersection of the strands), the thickness of the material, and the spacing between the strands. For example, depending on the angle of the weave, the degree of expansion and anchoring forces may vary. And, depending on the degree of expansion and anchoring forces exerted onto the wall of the inside surface of the coronary sinus 20, which results in reshaping of the wall, the diameter and the length of the coronary sinus 20 will gradually change over a period of time. For example, a smaller angle of weave (i.e., tight weaving) generally exerts greater expansion force as the elongate body 1300 expands. Moreover, due to its spring-like configuration, when the elongate body 1300 is compressed along the longitudinal axis of the elongate body 1300, the angle of the weave also tightens or reduces, preferably close to 0 degrees. However, when the elongate body 1300 is released or expanded along the longitudinal axis of the elongate body 1300, the angle of the weave expands, for example, in the range of 45 to 90 degrees radially along the longitudinal axis, to retain its original shape. As the angle of the weave expands further in the radial direction along the longitudinal axis of the elongate body 1300, the expansion force weakens.

With regard to the thickness of the material, thicker material exerts greater expansion force as the elongate body 1300 transforms from its compressed state to the expanded state. With regard to the spacing between the strands, smaller spacing between the strands requires a greater number of strands in the elongate body, resulting in greater expansion force as the elongate body 1300 transforms from its compressed state to the expanded state. At the same time, it is important to select a material and control the above-mentioned factors to ensure a smooth surface of the elongate body 1300 that minimizes trauma to the coronary sinus 20.

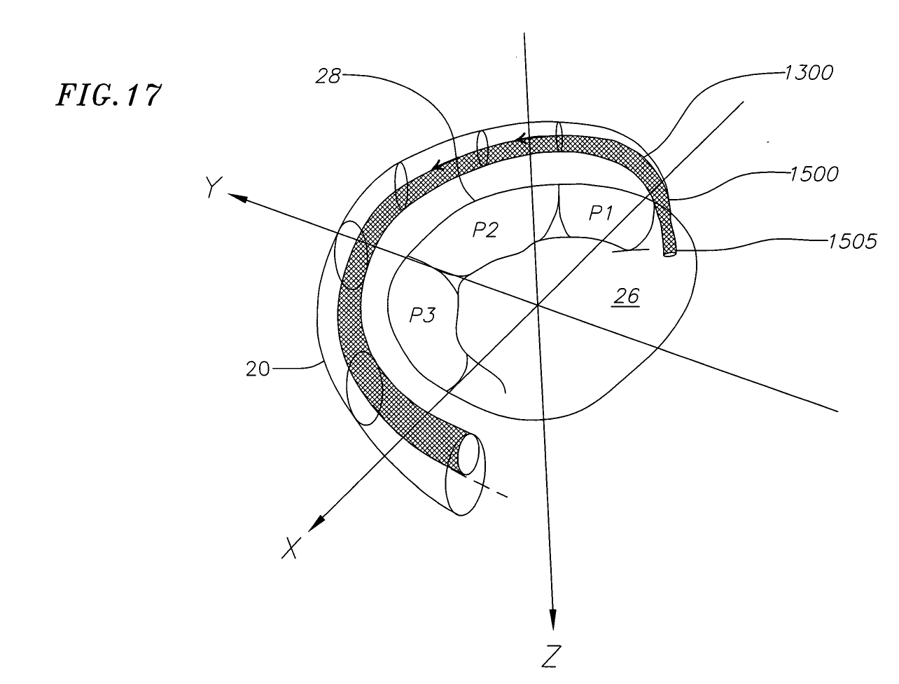

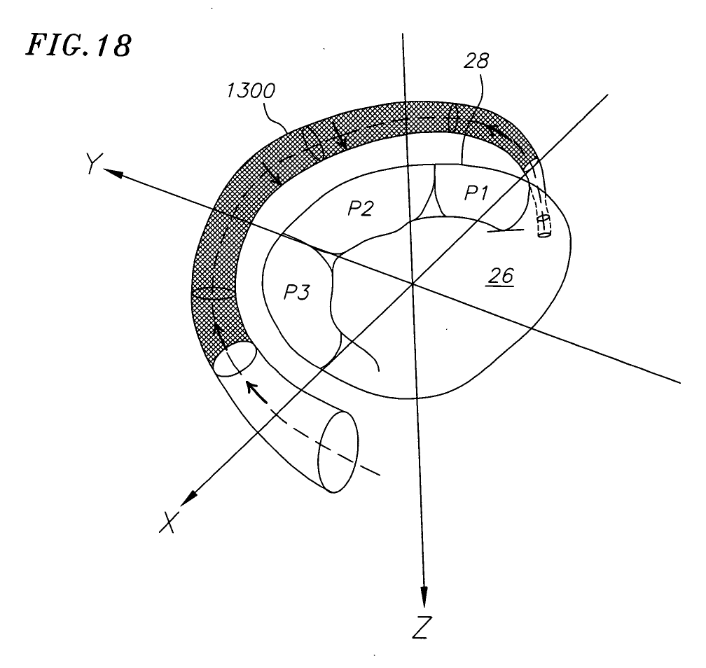

As briefly mentioned above, the elongate body 1300 has two states, a compressed state and an expanded state, as shown in FIGS. 17 and 18, respectively. Referring to FIG. 17, in the compressed state, the elongate body 1300 is enclosed within a lumen 1505 of a sheath 1500 and is inserted into the coronary sinus 20 via the sheath 1500, which acts as a delivery catheter. The elongate body 1300, still enclosed within the lumen 1505 is positioned in the coronary sinus 20 so that the central stent section 1310 is generally aligned, for example, with the P2 scallop. In the compressed state, the elongate body 1300 has a diameter that has been compressed to fit into the lumen 1505 and is flexible enough to move with the sheath 1500 along the curvatures of the coronary sinus 20. In this state, the elongate body 1300 has a uniform diameter that ranges from about 1.5 to 4 mm as it is enclosed within the lumen 1505.

Referring to FIG. 18, the sheath is pulled from the elongate body 1300 to expose the elongate body 1300 to the walls of the coronary sinus 20 and to allow it to expand into a three-dimensional shape that conforms to the anatomy of the coronary sinus 20. As the elongate body 1300 expands, the strands of the weave of the three-dimensional shape make contact with the circumference of the coronary sinus 20 and the entire length of the elongate body 1300 anchors tightly onto the wall of the inside surface of the coronary sinus 20. In addition to the anchoring provided by the woven structure of the elongate body 20, the funnel-shaped flare ends and slight bend of the proximal and distal stent sections 1305, 1315 provide further anchoring of the elongate body 1300. In one embodiment, the flare end of the proximal stent section 1305 expands against the circumference of the coronary sinus ostium 24 and the flare end of the distal stent section 1315 expands against the circumference at the distal end of the coronary sinus 20.

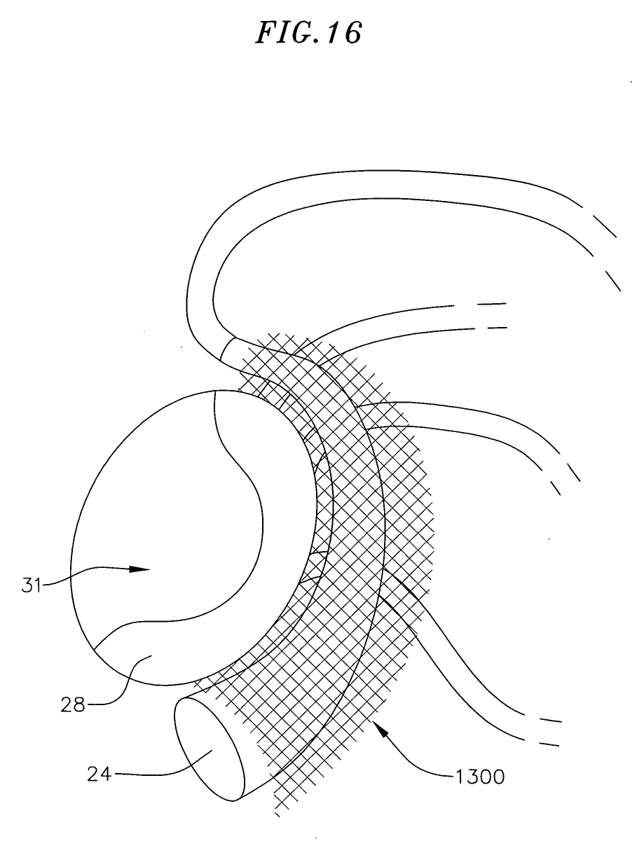

As discussed above, the elongate body 1300 is designed so that when it is expanded, it has a curved shape that follows the anatomical curvature of the coronary sinus 20 and makes substantial contact with the walls along the inside of the arcuate path of the coronary sinus 20. The expansion force of the elongate body 1300, which has been determined by various factors such as the angle of the weave, continues to push the walls of the coronary sinus 20 radially outward and pull the ends of the elongate body 1300 toward the central section 1310 of the elongate body 1300. Over a period of time, e.g. several weeks, the diameter elongate body continues to expand. As the elongate body 1300 expands, radially, it gradually grows through the wall of the coronary sinus 20 and attaches to scar tissue created by the elongate body's penetration of the wall of the coronary sinus (FIG. 16). Radial expansion of the elongate body 1300 through the wall of the coronary sinus 20 foreshortens the coronary sinus and also reduces the radius of curvature of the coronary sinus. Such changes in the coronary sinus 20 cinches the coronary sinus more tightly around the P1, P2 and P3 scallops of the mitral valve 26 and pushes one or more of the scallops, closer to the anterior leaflet 28 of the mitral valve. This allows a gap between the anterior leaflet 29 and the P1, P2 and P3 scallops of the posterior leaflet 31 to close and achieve remodeling of the mitral annulus 28 over the span of several weeks. When the gap between the mitral valve leaflets is closed, the effects of mitral valve regurgitation are drastically reduced or eliminated. The elongate body 1300 may be coated with antithrombogenic material to prevent thrombosis and occlusion of the coronary sinus, which may occur in the remodeling of the coronary sinus.

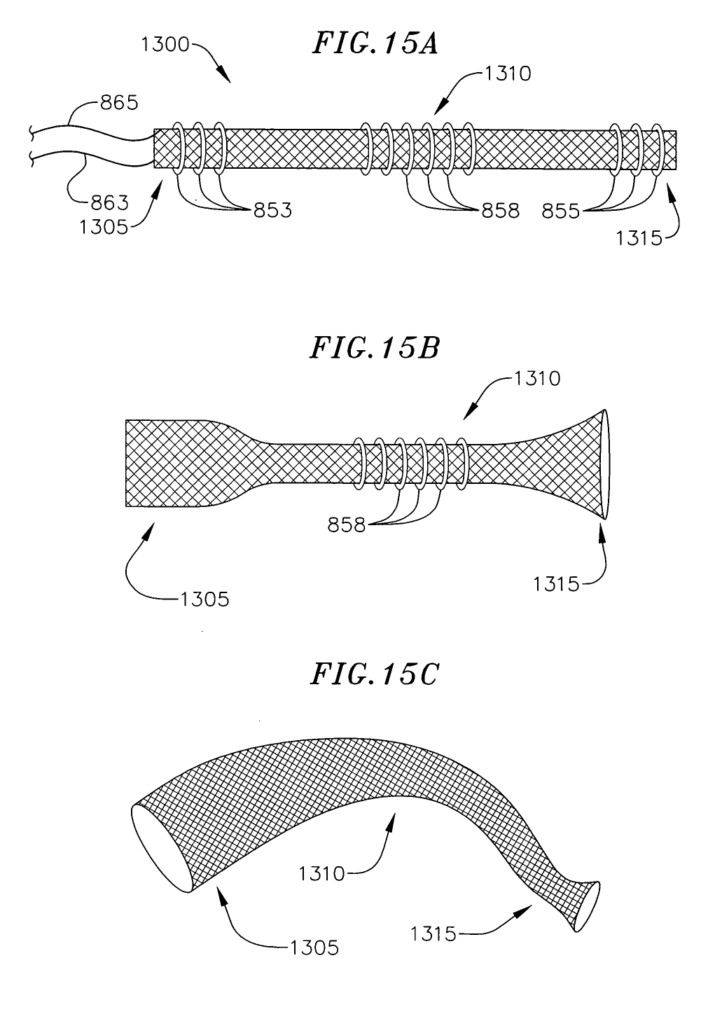

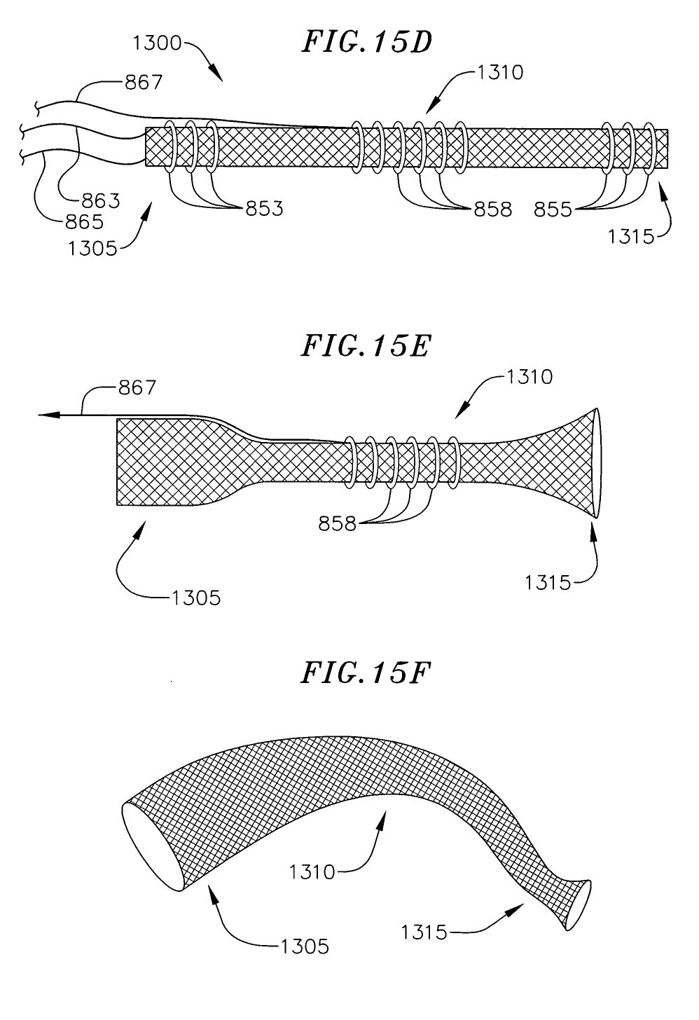

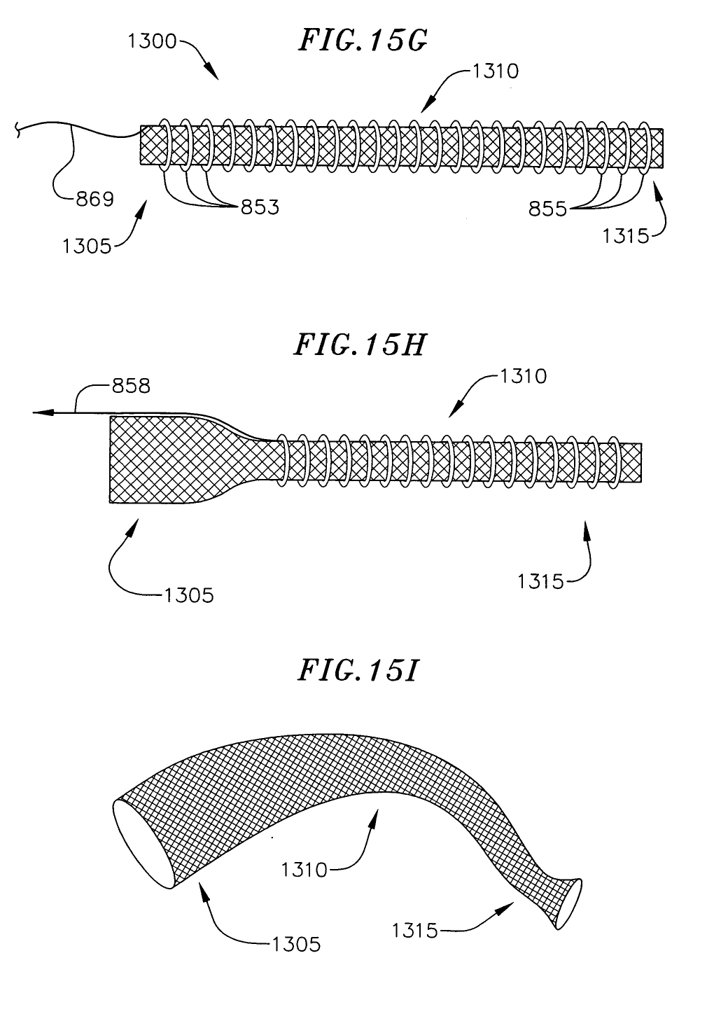

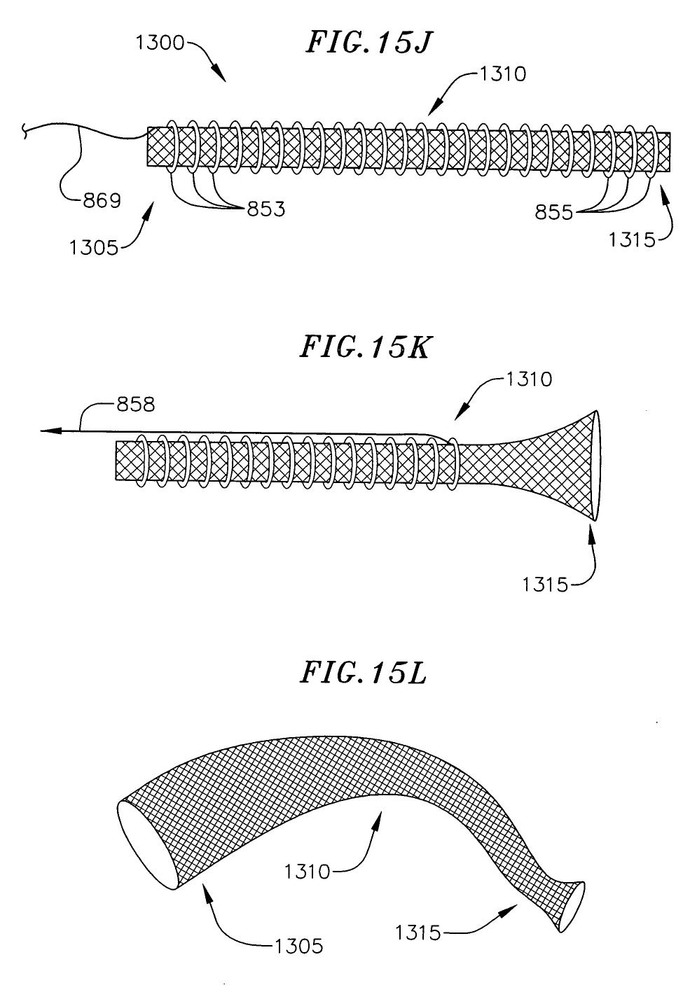

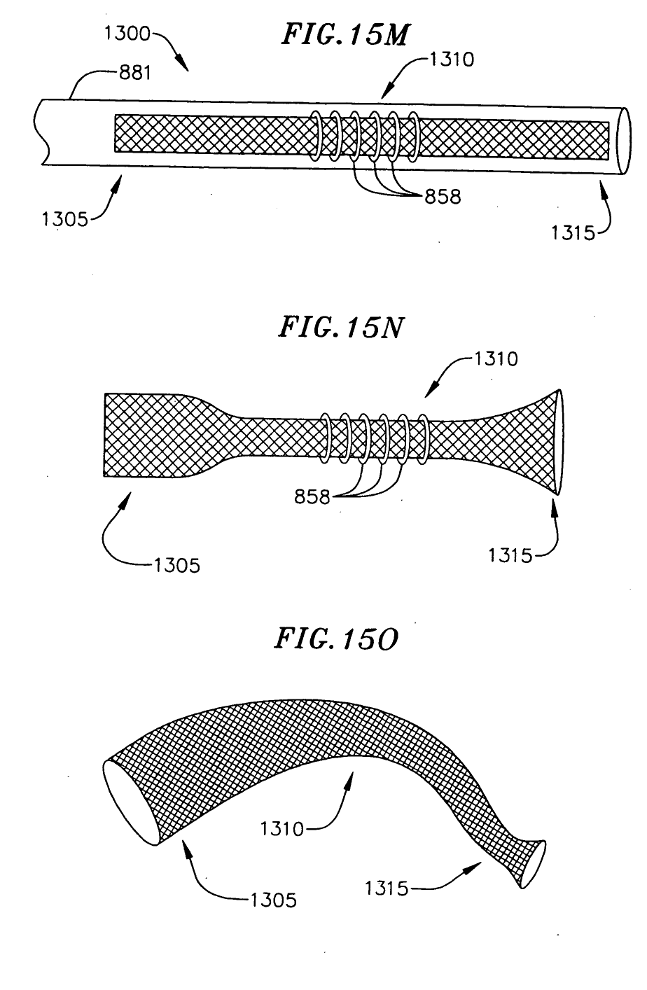

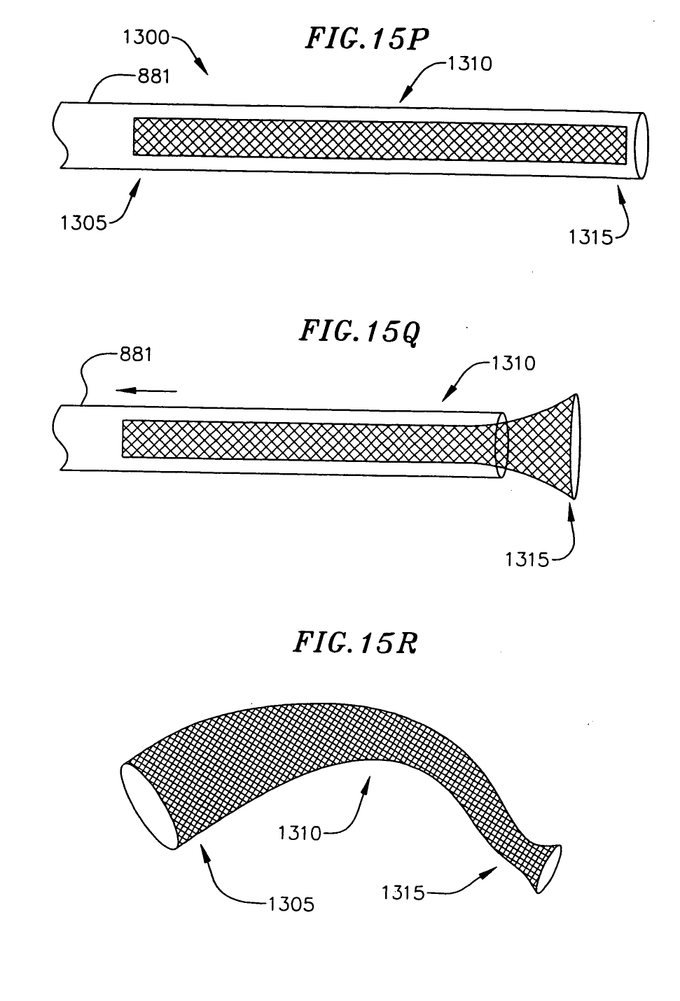

FIGS. 15A to 15S in general show various additional embodiments of the present invention.

Referring now to FIGS. 15A-15C, a further alternative embodiment of the present invention is described, in which the device comprises a tapered stent having proximal and distal sections that are joined by a central section capable of assuming a predetermined curvature. In FIG. 15A, elongate body 1300 includes a wire mesh stent having proximal stent section 1305, distal stent section 1315 and central stent section 1310, and is designed to conform to the taper of the coronary sinus. In FIG. 15A, the elongate body 1300 is shown in its elongated and radially crimped state. Elongate body 1300 is shown in its fully radially expanded and axially foreshortened state in FIG. 15C. Further in accordance with the principles of the present invention, elongate body 1300 includes one or more biodegradable structures 858, such as sutures, disposed on central stent section 1310 to retain that section in the contracted shape for a predetermined period after placement of the device in a patient's coronary sinus. Examples of biodegradable structures are described in more detail below.

Elongate body 1300 also includes at least one proximal retaining element 853 that retains proximal stent section 1305 in a contracted state, and further includes at least one distal retaining element 855 that retains distal stent section 1315 in a contracted state. Proximal and distal retaining elements 853 and 855 may comprise one or more sutures disposed about proximal and distal sections 1305 and 1315, respectively. Proximal and distal retaining elements 853 and 855 may be coupled to distal ends of strands 863 and 865, respectively. A physician may actuate strands 863 and 865, e.g., by retracting proximal ends of the strands, to deploy proximal and distal sections 1305 and 1315, respectively, as shown in FIG. 15B.

Proximal and distal sections 1305 and 1315 may comprise a shape-memory alloy, such as Nitinol, that self-expands to a predetermined shape when retaining elements 853 and 855 are removed.

In another embodiment of the present invention as shown in FIGS. 15D-15F, the central stent section 1310 of the elongate body 1300 delivered in a restraining catheter has a restraining thread 867 extending outside of the vasculature and the patient to be retracted by the physician at the desired time. Retraction of the restraining thread 867 will allow the central section 1310 of the elongate body 1300 to expand radially.

Additionally, as shown in FIGS. 15G-15I, a single restraining thread 869 may cover the entire elongate body 1300. The thread may be wrapped around the elongate body 1300 in such a way that, when it is retracted by the physician, it unravels from the proximal end 1305 to the distal end 1315 of the elongate body 1300. Alternatively, as shown in FIGS. 15J-15L, the single restraining thread 869 may be wrapped around the elongate body 1300 in such a way that, when it is retracted by the physician, it unravels from the distal end 854 to the proximal end 152 of the elongate body 1300. Such restraint, as described by at least the last two embodiments, makes a restraining catheter unnecessary. Alternatively, retaining elements 853 and 855 may be omitted, and proximal and distal sections 1305 and 1315 may self-expand to the predetermined shape upon retraction of a constraining sheath.

In yet another embodiment of the present invention, as shown in FIGS. 15M-15P, a restraining catheter 881 is placed over the elongate body 1300 before the device is inserted into a patient. Additionally, a biodegradable restraining thread 858 is placed around the central stent section 1310 of the elongate body 1300. When the restraining catheter 881 is removed, the proximal and distal stent sections 1305, 1315 of the elongate body 1300 expand immediately, while the central stent section 1310 will expand over time as the restraining thread 858 is absorbed by the body. Alternatively, as shown in FIGS. 15Q-15S, only a restraining catheter 881 is placed over the elongate body 1300. Thus, as the restraining catheter is retracted, the elongate body 1300 expands immediately from the distal end 1315 to the proximal end 1305.

In one exemplary embodiment, all three sections 1305, 1310, 1315 of the stent are integrally formed from a single shape memory alloy tube, e.g., by laser cutting. The sections 1305, 1310, 1315 are then processed, using known techniques, to form a self-expanding unit. In another embodiment, the device may be braided from Nitinol, stainless steel or other metal alloy threads and cut to the appropriate length. Such braiding permits the creation of three-dimensional shapes, allowing the device to more closely conform to the shape of the coronary sinus.

Unlike some of the preceding embodiments, which rely upon drawing proximal and distal elements together at the time of deploying the device, this embodiment of the present invention permits proximal and distal sections 1305 and 1315 to become biologically anchored in the venous vasculature before those sections are drawn together by expansion and/or curvature of central stent section 1310 to remodel the mitral valve annulus.

The elongate body 1300 may be deployed as follows. Elongate body 1300 is loaded into a delivery sheath and positioned within the patient's coronary sinus. The delivery sheath then is retracted proximally to expose distal stent section 1315, as shown in FIG. 15B. Distal stent section 1315 may be deployed when the proximal end of strand 865, which is coupled to retaining element 855, is actuated by a physician. Alternatively, retaining element 855 may be omitted and distal stent section 1315 may self-expand upon retraction of the delivery sheath. Upon deployment using either technique, distal stent section 1315 radially expands to engage the intima of the coronary sinus.

The delivery sheath is then further proximally retracted to expose proximal stent section 1305 as shown in FIG. 15B. Proximal stent section 1305 may be deployed when strand 863, which is coupled to retaining element 853, is actuated by a physician. Alternatively, retaining element 853 may be omitted and proximal stent section 1305 may self-expand upon further retraction of the delivery sheath. Upon deployment using either technique, proximal stent section 1305 radially expands to engage the intima of the coronary sinus.

At the time of deployment of proximal and distal sections 1305 and 1315, central stent section 1310 is retained in a contracted state by biodegradable structures 858, illustratively biodegradable sutures, e.g., a poly-glycol lactide strand or VICREL suture, offered by Ethicon, Inc., New Brunswick, N.J., USA.

Over the course of several weeks to months, proximal and distal sections 1305 and 1315 of the stent will endothelialize, i.e., the vessel endothelium will form a layer that extends through the apertures in the proximal and distal sections of elongate body 1300 and causes those sections to become biologically anchored to the vessel wall. This phenomenon may be further enhanced by the use of a copper layer on the proximal and distal stent sections, as this element is known to cause an aggressive inflammatory reaction. Conversely, to reduce thrombosis on the central stent section 1310 of the stent 850, the central section and associated structures may be coated with an anticoagulant material. As a further alternative, the central section of the stent may be coated with a taxol derivative or other elutable drug.

Over the course of several weeks to months, or after the proximal and distal sections have become anchored in the vessel, biodegradable structures 858 that retain central stent section 1310 in the contracted state will biodegrade. Eventually, the self-expanding force of the central section will cause the biodegradable structures to break, and release central stent section 1310. Because central stent section 1310 is designed to assume a predetermined curvature as it expands radially, it causes the proximal and distal sections 1305 and 1315 of elongate body 1300 to curve accordingly, resulting in the fully deployed shape depicted in FIG. 15C. The forces created by expansion and curvature of central stent section 1310 thereby compressively loads, and thus remodels, the mitral valve annulus.

In an alternative embodiment, as shown in FIG. 16, the elongate body 1300 is “oversized.” In other words, the elongate body 1300 is manufactured deliberately to be larger than the natural size of the coronary sinus, even in the coronary sinus' most expanded state. Thus, as the elongate body 1300 expands, it slowly passes through the wall of the coronary sinus, causing the coronary sinus to form tissue and grow around the device. Since the device “outgrows” the coronary sinus, additional foreshortening may be achieved and the mitral valve annulus will be able to be more remodeled than with an ordinary sized device.

Biodegradable sutures may be designed to rupture simultaneously, or alternatively, at selected intervals over a prolonged period of several months or more. In this manner, progressive remodeling of the mitral valve annulus may be accomplished over a gradual period, without additional interventional procedures. In addition, because the collateral drainage paths exist for blood entering the coronary sinus, it is possible for the device to accomplish its objective even if it results in gradual total occlusion of the coronary sinus.

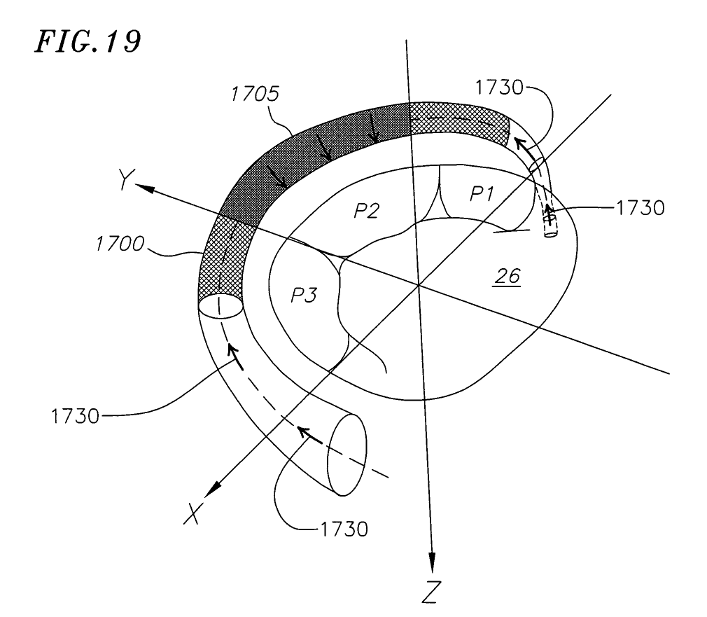

Another embodiment of the present invention, as shown in FIG. 19, comprises an outer elongate body 1700 and a rigid inner elongate body 1705 placed inside of the outer elongate body 1700 and eventually tightly fitted onto the wall of the inside surface of the outer elongate body 1700. The outer elongate body 1700 is flexible such that it can evenly distribute the expansion forces along the wall of the coronary sinus 20 during the foreshortening of the coronary sinus 20. For example, elongate body 1300 described in FIG. 13 may be used. The rigid inner elongate body 1705, which is placed inside of the outer elongate body 1700 and has the length in the range of 30 mm to 80 mm in its unstressed state, provides higher radial strength and rigidity to further straighten the coronary sinus 20 and to exert greater force onto the mitral annulus 28, in addition to the foreshortening provided by the outer elongate body 1700 (shown by the arrows 1730 in FIG. 19). To provide sufficient rigidity with an effective straightening effect, the inner elongate body 1705 is made of a rigid metal, such as stainless steel. In one configuration, the inner elongate body 1705 is a tubular structure made of stainless steel in a mesh configuration. The mesh configuration includes a series of connected stainless steel loops, each loop having a zigzag shape with peaks. For example, the elongate body 10 described in FIG. 2 may be used.

The two elongate bodies 1700, 1705 are deployed with separate delivery means. First, the outer elongate body 1700, which may be self-expandable, as described with respect to the elongate body 1300 of FIGS. 13 and 14, or balloon-expandable, is deployed and placed into the coronary sinus 20 as shown in FIG. 19. The expansion of the outer elongate body 1700 results in foreshortening of the coronary sinus 20, which in turn results in reshaping of the mitral annulus 28.

Next, the inner elongate body 1705, which may be self-expandable or balloon-expandable, is deployed and placed inside of the inner surface of the outer elongate body 1700. In one configuration, the inner elongate body 1705 is deployed with a balloon. In this configuration, the inner elongate body 1705 is mounted onto a balloon (not shown), which acts as a delivery catheter. Once the inner elongate body 1705 and the balloon are appropriately positioned inside of the outer elongate body 1700, the balloon is expanded by introducing, for example, a saline solution through the delivery catheter and into the balloon. Alternately, any biocompatible solution may be used to inflate the balloon. Once the inner elongate body 1705 is expanded to make substantial contact with the outer elongate body 1700 and is tightly fitted along the walls of the inside surface of the outer elongate body 1700, the balloon is deflated and removed. Depending on the location of the regurgitation jet in the mitral valve, the rigid inner elongate body 1705 can be placed anywhere along the wall of the coronary sinus 20 that aligns with the posterior section of the mitral annulus 28 to further increase the effect of the inward displacement of the mitral annulus 28 (as shown by the arrows of FIG. 19). Typically, the inner elongate body 1705 is placed within the central stent section of the outer elongate body 1700 to straighten the central section of the coronary sinus 20, which is generally aligned with the P2 scallop.

Resorbable materials have been used in connection with valve repair devices as a means to provide a “delayed release” mechanism allowing a device to effect a change to a valve over time. Examples of embodiments that include resorbable material may be found in U.S. patent application Ser. Nos. 10/141,348 to Solem, et al., 10/329,720 to Solem, et al., and 10/500,188 to Solem, et al., which are incorporated herein by reference.

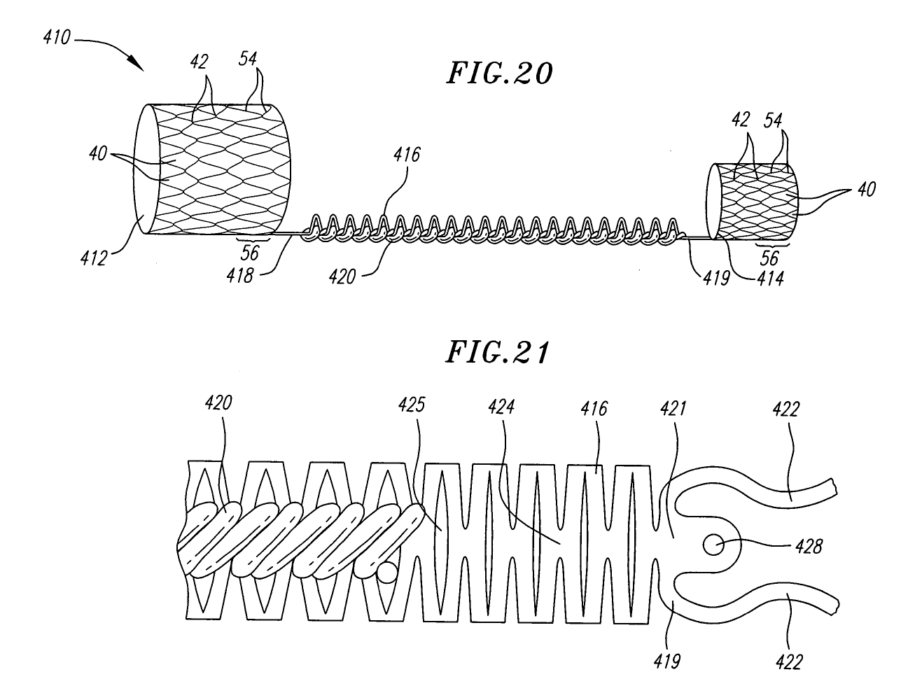

As shown in FIG. 20, a new embodiment of the present invention includes an elongate body 410 having resorbable thread sutured through the openings of a bridge 416. The elongate body further includes a proximal anchor 412 and a distal anchor 414 connected by the bridge 416 with the resorbable material.

Resorbable materials are those that, when implanted into a human body, are resorbed by the body by means of enzymatic degradation and also by active absorption by blood cells and tissue cells of the human body. Examples of such resorbable materials are PDS (Polydioxanon), Pronova (Polyhexafluoropropylen-VDF), Maxon (Polyglyconat), Dexon (polyglycolic acid) and Vicryl (Polyglactin). As explained in more detail below, a resorbable material may be used in combination with a shape memory material, such as nitinol, Elgiloy or spring steel to allow the superelastic material to return to a predetermined shape over a period of time.

In one embodiment as shown in FIG. 20, the proximal and distal anchors 412, 414 are both generally cylindrical and are both made from tubes of shape memory material, for example, nitinol. However, the anchors 412 and 414 may also be made from any other suitable material, such as stainless steel. Both anchors 412, 414 have a mesh configuration comprising loops 54 of zigzag shaped shape memory material having alternating peaks 42. The loops 54 are connected at each peak 42 to form rings 56 of four-sided openings 40. Other configurations may also be used as known in the art. Additionally, other types of anchors known in the art may also be used.

The proximal and distal anchors 412, 414 each have a compressed state and an expanded state. In the compressed state, the anchors 412, 414 have a diameter that is less than the diameter of the coronary sinus 20. In this state, the anchors 412 and 414 have a substantially uniform diameter of between about 1.5 to 4 mm. In the expanded state, the anchors 412, 414 have a diameter that is about equal to or greater than a diameter of the section of a non-expanded coronary sinus 20 to which each anchor will be aligned. Since the coronary sinus 20 has a greater diameter at its proximal end than at its distal end, in the expanded state the diameter of the proximal anchor 412 is between about 10-15 mm and the diameter of the distal anchor is between about 3-6 mm.

In one embodiment, the bridge 416 is connected between the proximal anchor 412 and distal anchor 414 by links 418, 419. More specifically as shown in FIG. 20, a proximal link 418 connects the proximal stent section 412 to a proximal end of the bridge 416 and a distal link 419 connects the distal stent section 414 to a distal end of the bridge 416. The links 418 and 419 have a base 421 and arms 422 that extend from the base and which are connected to two peaks 42 on each anchor 412, 414. Further, the links 418 and 419 contain a hole 428, as shown in FIG. 21, which serves as a means through which to pass the end of the resorbable thread and secure it to the bridge 416.

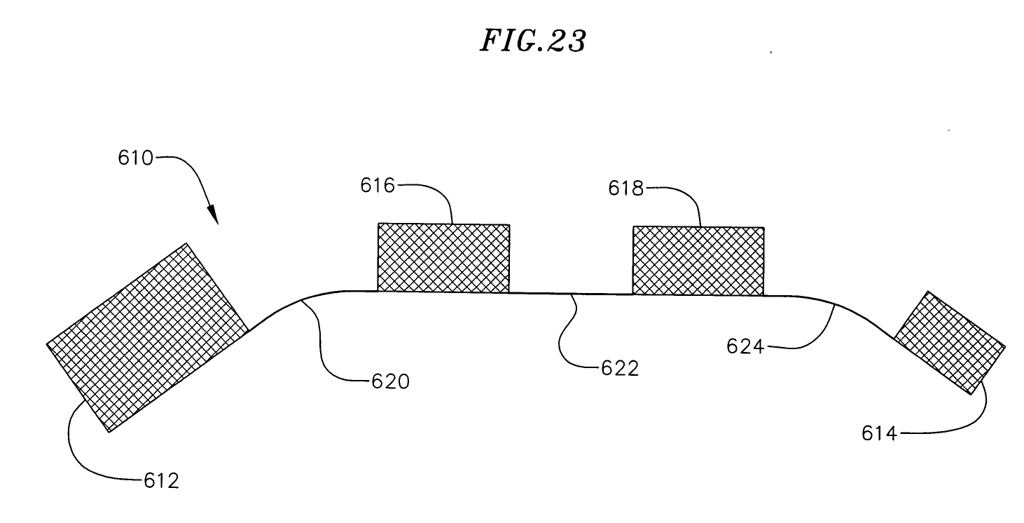

The bridge 416 in one embodiment is made from a shape memory material and is flexible to allow the body 410 to conform to the shape of the coronary sinus 20. The bridge 416 comprises X-shaped elements 424 wherein each X-shaped element is connected to an adjacent X-shaped element at the extremities of the “X,” allowing a space 425 to be created between adjacent X-shaped elements, as shown in FIG. 23. The X-shaped elements 424 further have rounded edges that minimizes the chances that a sharp edge of the bridge 416 will puncture or cut a part of the coronary sinus 20 as the device is inserted. The bridge 416 has two states: an elongated state in which the bridge 416 has a first length, and a shortened state in which the bridge has a second length, the second length being shorter than the first length. In the present embodiment, resorbable thread 420 is woven into the spaces 425 between adjacent X-shaped elements 424 to hold the bridge 416 in its elongated state. The thread 420 acts as a temporary spacer. When the resorbable thread 420 is dissolved over time by means of resorption, the bridge assumes its shortened state.

The present embodiment is deployed as follows. An introduction sheath (not shown) made of synthetic material is used to gain access to the venous system. A guide wire (not shown) is then advanced through the introduction sheath and via the venous system to the coronary sinus 20. The guide wire and/or introduction sheath is provided with radiopaque distance markers which can be identified using X-rays which allows the position of the body 410 in the coronary sinus 20 to be monitored.

The elongate body 410 is mounted onto a stent insertion device (not shown) so that the self-expanding anchors 412 and 414 are held in the compressed state. Thereafter, the stent insertion device with the elongate body 410 mounted thereon is pushed through the introduction sheath and the venous system to the coronary sinus 20 riding on the guide wire. After the body 410 is positioned in the coronary sinus 20 so that the center of the body is generally aligned with the center of the P2 scallop, the stent insertion device is removed. When the stent insertion device is removed, the self-expandable anchors 412 and 414 are released so that they expand and contact the inner wall of the coronary sinus 20 and provide temporary fixation of the elongate body 410 to the coronary sinus. Alternatively, the anchor may be expanded by balloons or other means known in the art. In one embodiment, the device can be rotated so that the bridge contacts the wall of the coronary sinus that is closest to the mitral valve 26. The guide wire and the introduction sheath are then removed.

After the body 410 is inserted into the coronary sinus 20, the wall of coronary sinus will grow around the mesh configuration of the anchors 412 and 414. Simultaneously, the resorbable thread 420 will be resorbed by the surrounding blood and tissue in the coronary sinus 20. After a period of a few weeks, the anchors 412 and 414 will be secured into the wall of the coronary sinus 20. During that time period, the resorbable thread 420 will be resorbed to such a degree that eventually it can no longer hold the bridge 416 in its elongated state. As the resorbable thread 420 is resorbed, the bridge 416 retracts from its elongated state to its shortened state. This shortening of the bridge 416 draws the proximal anchor 412 and the distal anchor 414 together, cinching the coronary sinus 20 and/or reducing its circumference. This cinching and/or reduction of the circumference of the coronary sinus 20 closes the gap created by dilatation of the posterior leaflet 31 of the mitral valve.

The body 410 may be positioned in the coronary sinus 20 by catheter technique or by any other adequate technique. The body 410 may be heparin-coated so as to avoid thrombosis in the coronary sinus 20, thus reducing the need for aspirin, ticlopedine or anticoagulant therapy. At least part of the body 410 may contain or be covered with any therapeutic agents such as Tacrolimus, Rappamycin or Taxiferol to prohibit excessive reaction with surrounding tissue. Further, at least parts of the body 410 may contain or be covered with Vascular Endothelial Growth Factor (VEGF) to ensure smooth coverage with endothelial cells.

In some cases of ischemic mitral regurgitation, the dilatation of the mitral annulus may be asymmetric with, for example, one region of the mitral annulus being more dilated than another. Thus, it may be advantageous to be able to control the degree of cinching along a particular segment of the mitral annulus.

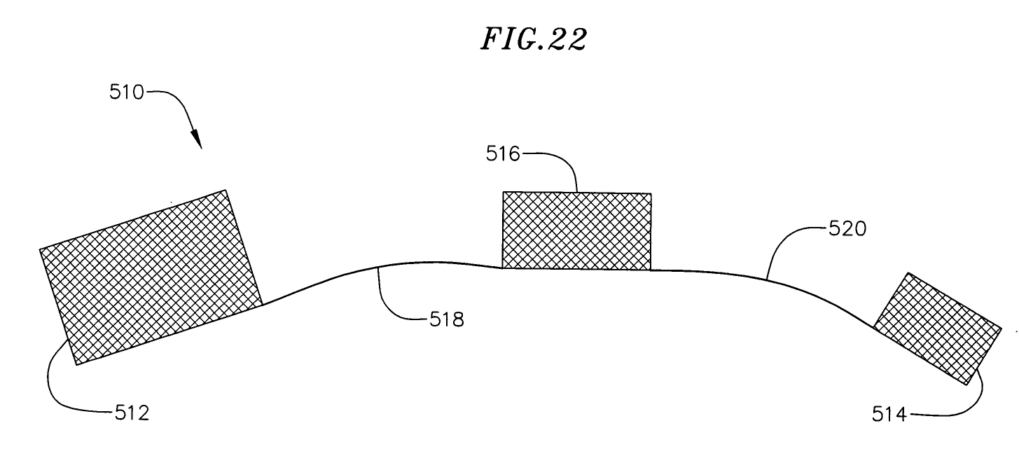

As shown in FIG. 22, an alternate embodiment of the present invention similar to the delayed release device described above comprises an elongate body 510 including a proximal anchor 512, a distal anchor 514 and a central anchor 516. A first bridge 518 connects the proximal anchor 512 to the central anchor 516, and a second bridge 520 connects the distal anchor 514 to the central anchor.

The structure of the elongate body 510 is substantially similar to the structure of the elongate body 410 described above. More specifically, each anchor 512, 514, 516 is generally cylindrical and has a compressed state and an expanded state. Further, each bridge 518, 520 has an elongated and a shortened state and comprises X-shaped elements with resorbable thread woven into spaces created between adjacent X-shaped elements. Also, each bridge 518, 520 is connected to its respective anchors 512, 514, 516 by a link as described above.

The amount of foreshortening of the bridge 518 may be variable depending on, for example, the size of the X-shaped elements, the size of the openings between adjacent X-shaped elements, the type of material used to manufacture the bridge, and the diameter of the material threaded into the bridge.

The present embodiment is deployed as follows. An introduction sheath (not shown) made of synthetic material is used to gain access to the venous system. A guide wire (not shown) is then advanced through the introduction sheath and via the venous system to the coronary sinus 20. The guide wire and/or introduction sheath is provided with X-ray distance markers so that the position of the body 510 in the coronary sinus 20 may be monitored.

The elongate body 510 is mounted onto a stent insertion device (not shown) so that the self-expanding anchors 512, 514 and 516 are held in the compressed state. Thereafter, the stent insertion device with the elongate body 510 mounted thereon is pushed through the introduction sheath and the venous system to the coronary sinus 20 riding on the guide wire. After the body 510 is positioned in the coronary sinus 20 so that the central anchor 516 is generally aligned with the center of the P2 scallop, the stent insertion device is removed. When the stent insertion device is removed, the self-expandable anchors 512, 514 and 516 are released so that they expand and contact the inner wall of the coronary sinus 20 and provide temporary fixation of the elongate body 510 to the coronary sinus. In one embodiment, the device may be rotated so that the bridges contact the wall of the coronary sinus that is closest to the mitral valve 26. The guide wire and the introduction sheath are then removed.

After the body 510 is inserted into the coronary sinus 20, the wall of coronary sinus will grow around the mesh configuration of the anchors 512, 514 and 516. Simultaneously, the resorbable thread (not shown in detail) will be resorbed by the surrounding blood and tissue in the coronary sinus 20. After a period of a few weeks, the anchors 512, 514 and 516 will be more permanently secured into the wall of the coronary sinus 20. During that time period, the resorbable thread will be resorbed to such a degree that eventually it will not hold the bridges 518, 520 in their elongated state any longer. As the resorbable thread is resorbed, the bridges 518, 520 retract from their elongated state to their shortened state. This shortening of the bridges 518, 520 draws the proximal and distal anchors 512, 514 toward each other, cinching the coronary sinus 20 and reducing its circumference. The reduction of the circumference of the coronary sinus 20 closes the gap created by dilatation of the posterior leaflet 31 of the mitral valve.

Having the central anchor 520 between the proximal and distal anchors 512, 514 may allow for a different amount of foreshortening between each pair of adjacent anchors, depending on the length of the bridges 518, 520. Thus, the elongate body 510 may be more specifically tailored to reshape the mitral annulus according to a patient's needs. For example, the bridge between the proximal anchor 512 and central anchor 516 may shorten more than the bridge between the distal anchor 514 and the central anchor or vice versa. Further, having an additional anchor serves to improve the distribution of forces that act on the proximal and distal stents as well as improving the distribution of the forces that the bridges exert on the inner wall of the coronary sinus.

The delayed release device described above is not limited to three anchors. FIG. 23 shows an embodiment 610 of the present invention wherein four anchors 612, 614, 616, 618 and three bridges 620, 622, 624 are used, but it will be apparent to one skilled in the art that any number of anchors may be used and that the length of the bridges between each anchor may vary.

In addition to the embodiments described in detail above, those skilled in the art will appreciate other embodiments for connecting a proximal anchor, a distal anchor and at least one central anchor. Some of those embodiments may include a thread of shape memory material held in an elongated state by a sheath of resorbable material, scissors-shaped memory material held in an elongated state by a sheath of resorbable material or by resorbable material in tension, a coil of shape-memory material wrapped around a tube of resorbable material, ribbons of resorbable material wrapped around a tube of shape memory material. See, for example, the embodiment in Ser. No. 10/500,188.

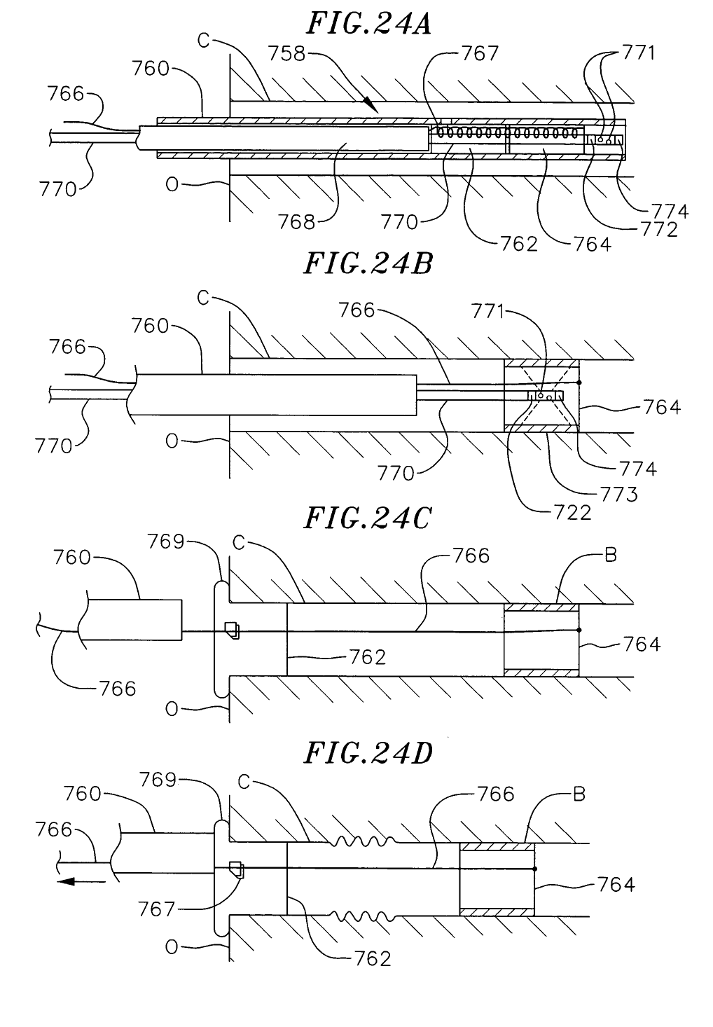

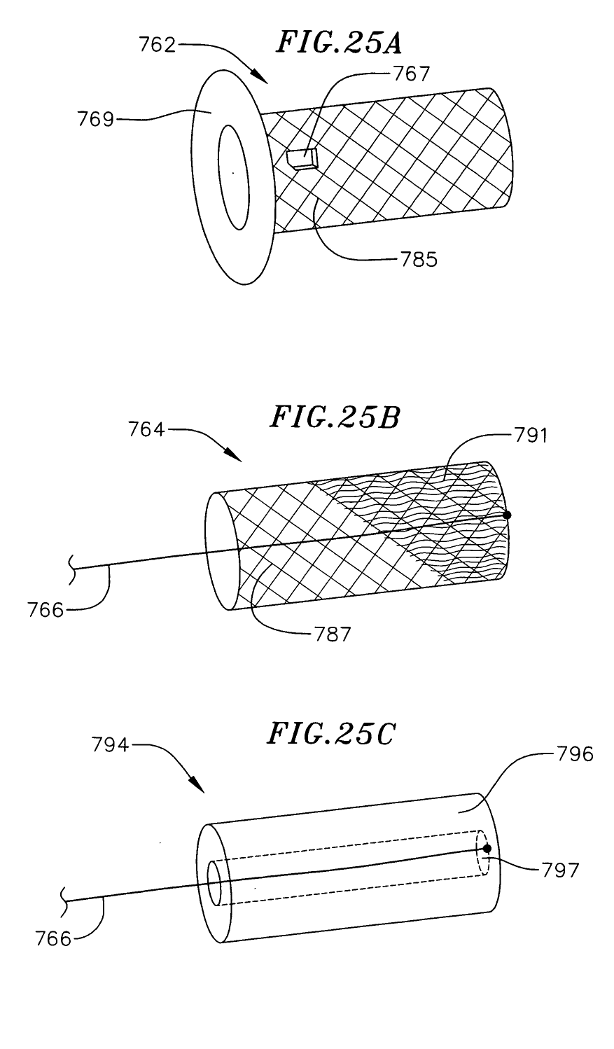

Referring now to FIGS. 24A-24D, another embodiment of the present invention is described. Apparatus 758 includes proximal anchor element 762 that is joined to distal anchor element 764 via wire 766 and cinch mechanism 767. Proximal and distal anchor elements 762 and 764 also include substantially tubular members that self-expand to engage the intima of the vessel in which they are deployed. In accordance with principles of the present invention, distal anchor element 764 includes a means for bonding the distal anchor element to at least a portion of an intima of coronary sinus C. Preferred configurations for proximal and distal anchor elements 762 and 764, as well as preferred means for bonding distal anchor element 764 to the intima of the coronary sinus, are described in detail with respect to FIGS. 25A-25C.

As shown in FIG. 25A, proximal anchor element 762 includes self-deploying stent 785 having proximal and distal ends, deployable flange 769 disposed at the proximal end, and cinch mechanism 767 coupled to stent 785. Stent 785 and deployable flange 769 of proximal anchor element 762 are initially constrained within delivery sheath 760, as shown in FIG. 24A, and are composed of a shape memory material, e.g., Nitinol, so that stent 785 and flange 769 self-deploy to the predetermined shapes shown in FIG. 25A upon retraction of delivery sheath 760.

Flange 769 may include a substantially circular shape-memory member, as illustrated in FIG. 25A, a plurality of wire members, e.g., manufactured using Nitinol, that self-deploy upon removal of sheath 764 and abut ostium O, or other suitable shape.

As shown in FIG. 25B, distal anchor element 764 preferably includes wire mesh stent 787 manufactured using a shape memory material, e.g., Nitinol. Wire 766 is coupled to distal anchor element 764 and is used in combination with cinch mechanism 767 of proximal anchor element 762 to remodel the coronary sinus, as described hereinbelow. Stents 785 and 787 are illustratively described as comprising wire mesh, but one of skill in the art will appreciate that other types of anchor elements including self-expanding slotted tubular stents also may be employed.

Distal anchor element 764, as depicted in FIG. 25B, in one exemplary embodiment is at least partially coated with a bonding material 791. Bonding material 791 may have light-reactive binding agents that undergo polymerization when exposed to radiation, for example, ultraviolet (UV) radiation. When bonding material 791 has such UV-curable agents, the agents may include acrylates, and more specifically, acrylates with UV or free radical polymerization or, for example, polymethylmethacrylate.

Apparatus 758 may further comprise catheter 770 having proximal and distal ends, a lumen extending therebetween, and at least one port 771 disposed at the distal end of the catheter, as shown in FIG. 24A. A light source, for example, including UV light, may be coupled to the proximal end of catheter 770 so that the light is transmitted throughout the lumen of catheter 770 and exits via port 771. Catheter 770 further includes radiopaque marker bands 772 and 774 to aid in the positioning of port 771 under fluoroscopy, which in turn ensures the proper positioning of the UV light.

Alternatively, bonding material 791 may include a synthetic molding material, such as a starch-based poly ethylene glycol hydrogel, that is heat hardenable or hydrophilic. In an exemplary embodiment, a starch-based poly ethylene glycol hydrogel is used that swells when exposed to an aqueous solution. Hydrogels also may be selected to harden, for example, upon exposure to body temperature or blood pH. Hydrogels suitable for use with the present invention may be obtained, for example, from Gel Med, Inc., Bedford, Mass.

Referring to FIG. 25C, alternative distal anchor element 794 may be used in lieu of distal anchor element 764 of FIG. 25B. Distal anchor element 794 includes foam member 796 having proximal and distal ends and bore 797 extending therebetween. Foam member 796 is depicted in a deployed state in FIG. 25C, but is capable of being contracted within delivery sheath 760 of FIG. 24A. Foam member 796 is made from a hydrophilic foam, i.e., a foam material that has a tendency to absorb water and swell into engagement with the vessel intima.

Referring back to FIG. 24A, preferred method steps for using the proximal and distal anchor elements of FIGS. 25A-25C are described. Apparatus 758 is navigated through the patient's vasculature with proximal and distal anchor elements 762 and 764 in a contracted state and into coronary sinus C, as shown in FIG. 24A. The distal end of sheath 760 is disposed, under fluoroscopic guidance, at a suitable position within the coronary sinus, great cardiac vein, or adjacent vein. Push tube 768 then is held stationary while delivery sheath 760 is retracted proximally so that distal anchor element 764 deploys from within sheath 760, thereby permitting distal anchor element 764 to self-expand into engagement with the vessel wall, as shown in FIG. 24B.

In accordance with principles of the present invention, after distal anchor element 764 self-deploys, an outer surface of distal anchor element 764 will become at least partially chemically or mechanically bonded to an intima of coronary sinus C. When bonding material 791 of FIG. 25B comprises a light-reactive binding agent, the light-reactive binding agents will at least partially contact the vessel wall when distal anchor element 764 self-deploys. At this time, light 773, for example, UV light, may be emitted from port 771 of catheter 770 to cause light-reactive agents 791 to polymerize, and thereby form bond B with the intima of coronary sinus C, as shown in FIG. 25B. Catheter 770 then may be removed upon satisfactory bonding of distal anchor element 764.

Alternatively, when bonding material 791 of FIG. 25B comprises a hydrogel, the exposure of the hydrogel to flow in the vessel will cause at least a portion of distal anchor element 764 to chemically bond with the intima of coronary sinus C. In yet another alternative embodiment, when alternative distal anchor element 794 of FIG. 25C is used, foam member 796 will cause distal anchor element 794 to chemically or mechanically bond with the intima of coronary sinus C when exposed to flow in the vessel due to the hydrophilic properties of foam member 796.

Using any of the techniques described above, it is possible to chemically bond distal anchor element 764, or distal anchor element 794, to at least a portion of the intima of coronary sinus C. As will be described in detail hereinbelow, this is advantageous because shear stress to the vessel will be reduced when actuating wire 766 and cinch mechanism 767.

Referring now to FIG. 24C, in a next method step, delivery sheath 760 is retracted proximally, under fluoroscopic guidance, until proximal anchor element 762 is situated extending from the coronary sinus. Push tube 768 is held stationary while sheath 760 is further retracted, thus releasing proximal anchor element 762. Once released from delivery sheath 760, proximal anchor element 762 self-expands into engagement with the wall of the coronary sinus C, and flange 769 abuts against coronary ostium O, as shown in FIG. 24C.

Delivery sheath 760 (and/or push tube 768) then may be positioned against flange 769 of proximal anchor element 762, and wire 766 retracted in the proximal direction to draw distal anchor element 764 towards proximal anchor element 762, as shown in FIG. 24D. As will of course be understood, distal anchor element 764 is drawn towards proximal anchor element 762 under fluoroscopic, ultrasound or other types of guidance, so that the degree of remodeling of the mitral valve annulus may be assessed.

As wire 766 is drawn proximally, cinch mechanism 767 prevents distal slipping of the wire. For example, wire 766 may include a series of grooves along its length that are successively captured in a V-shaped groove, a pall and ratchet mechanism, or other well-known mechanism that permits one-way motion. Upon completion of the procedure, delivery sheath 760 and push tube 768 are removed from the patient's vessel.

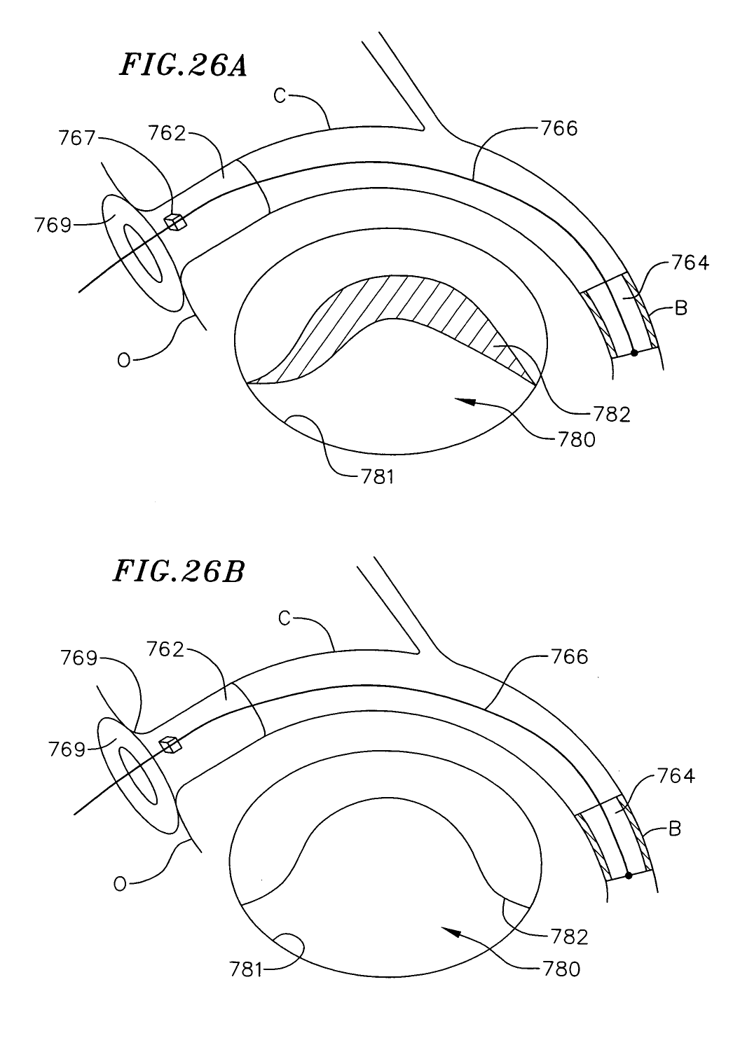

Referring now to FIGS. 26A-26D, a method for using apparatus 758 of FIGS. 6 and 7 to close a central gap 782 of mitral valve 780 is described. In FIG. 26A, proximal and distal anchor elements 762 and 764 are deployed in coronary sinus C, preferably so that flange 769 of proximal anchor element 762 abuts coronary ostium O. Distal anchor element 764 is disposed at such a distance apart from proximal anchor element 762 that the two anchor elements apply a compressive force upon mitral valve 780 when wire 766 and cinch 767 are actuated.

In FIG. 26B, cinch 767 is actuated from the proximal end to reduce the distance between proximal and distal anchor elements 762 and 764, e.g., as described hereinabove with respect to FIG. 24D. When wire 766 and cinch mechanism 767 are actuated, distal anchor element 764 is pulled in a proximal direction, while proximal anchor element 762 may be urged in a distal direction using delivery sheath 760 and/or push tube 768, as shown in FIG. 24D.

When proximal anchor element 762 comprises flange 769, proximal anchor element 762 is urged in the distal direction until flange 769 abuts coronary ostium O. The reduction in distance between proximal and distal anchor elements 762 and 764 reduces the circumference of mitral valve annulus 781 and thereby reduces gap 782. Flange 769 provides a secure anchor point that prevents further distally-directed movement of proximal anchor element 762, and reduces shear stresses applied to the proximal portion of the coronary sinus. Moreover, because distal anchor element 764 is bonded to the intima of coronary sinus C using any of the techniques described above, shear stress to the intima of coronary sinus C will be reduced when actuating wire 766 and cinch mechanism 767.

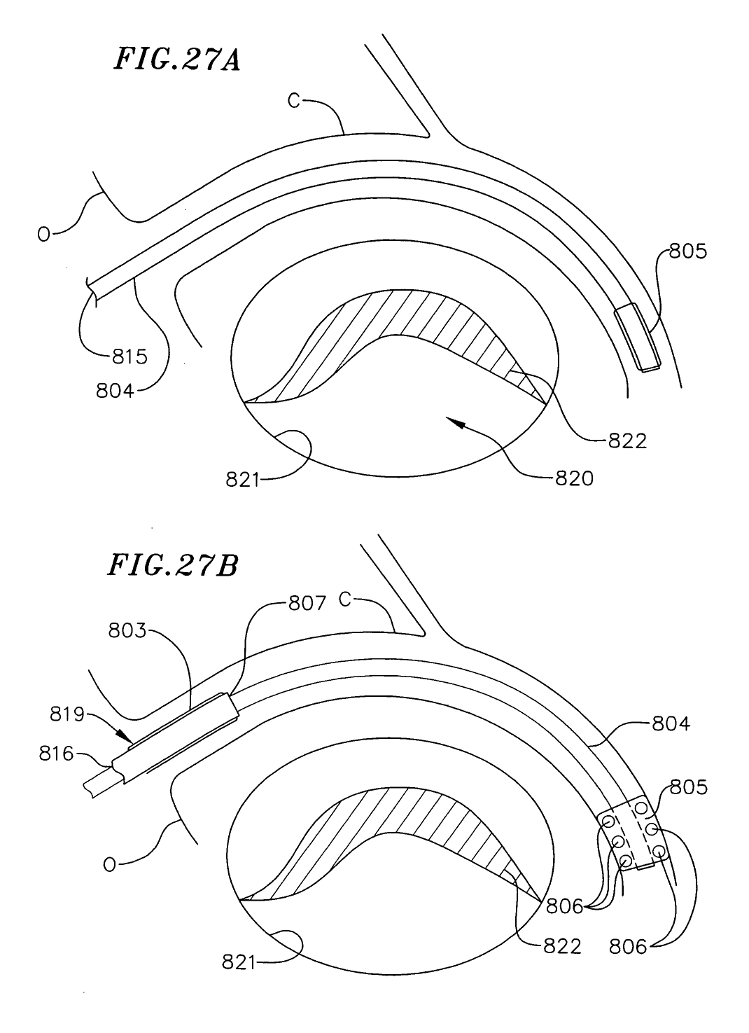

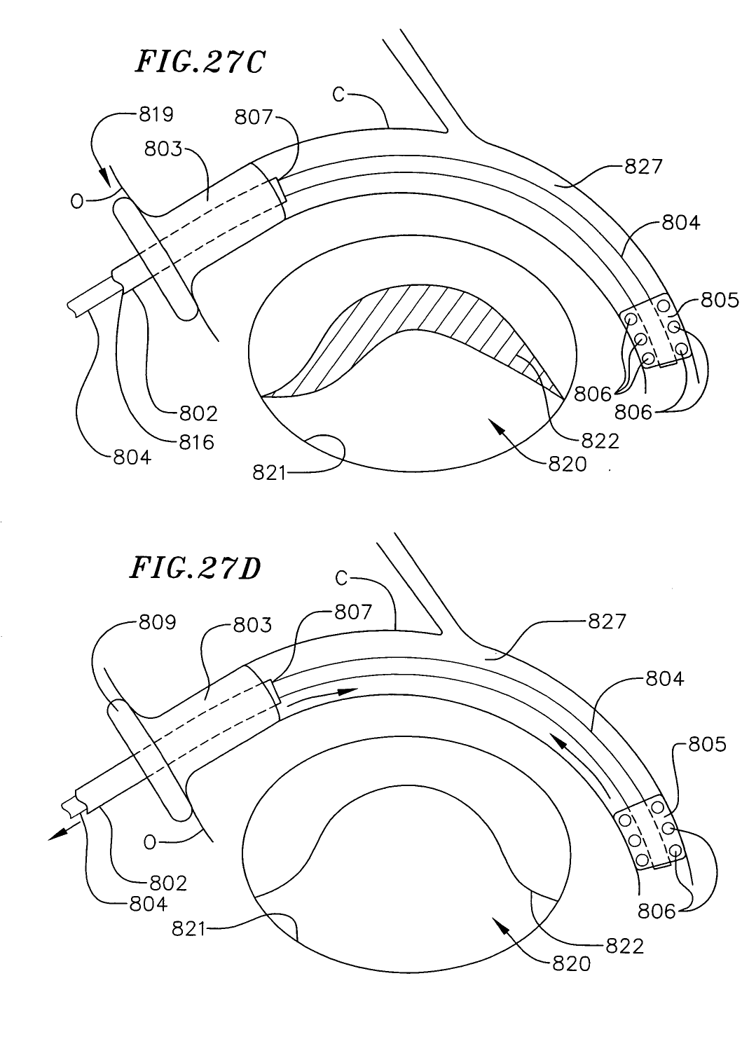

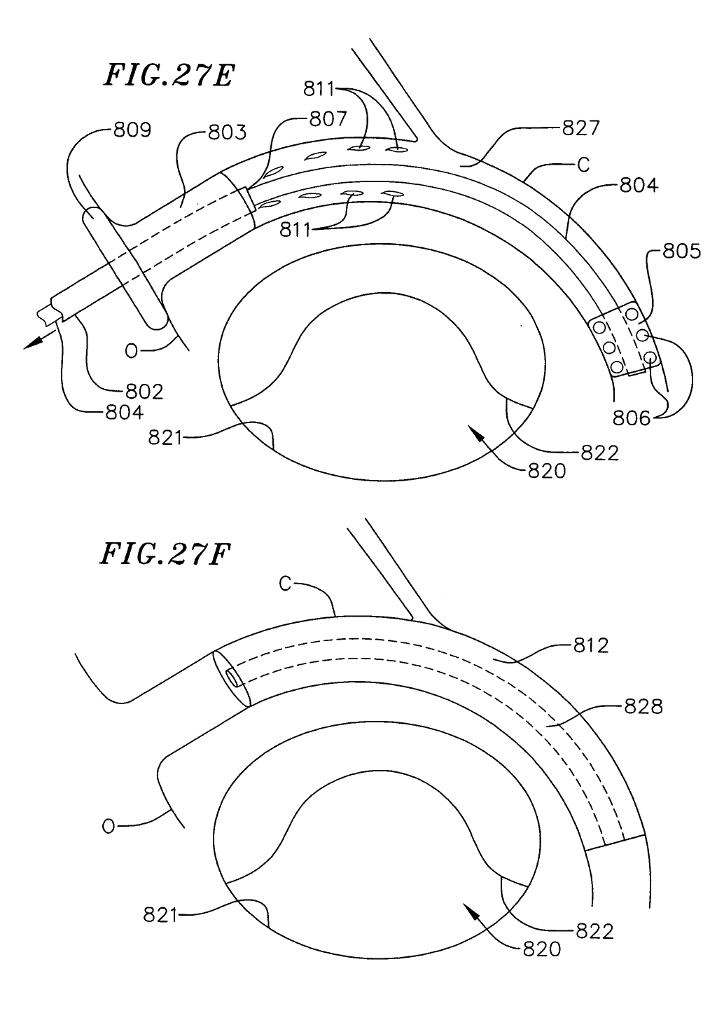

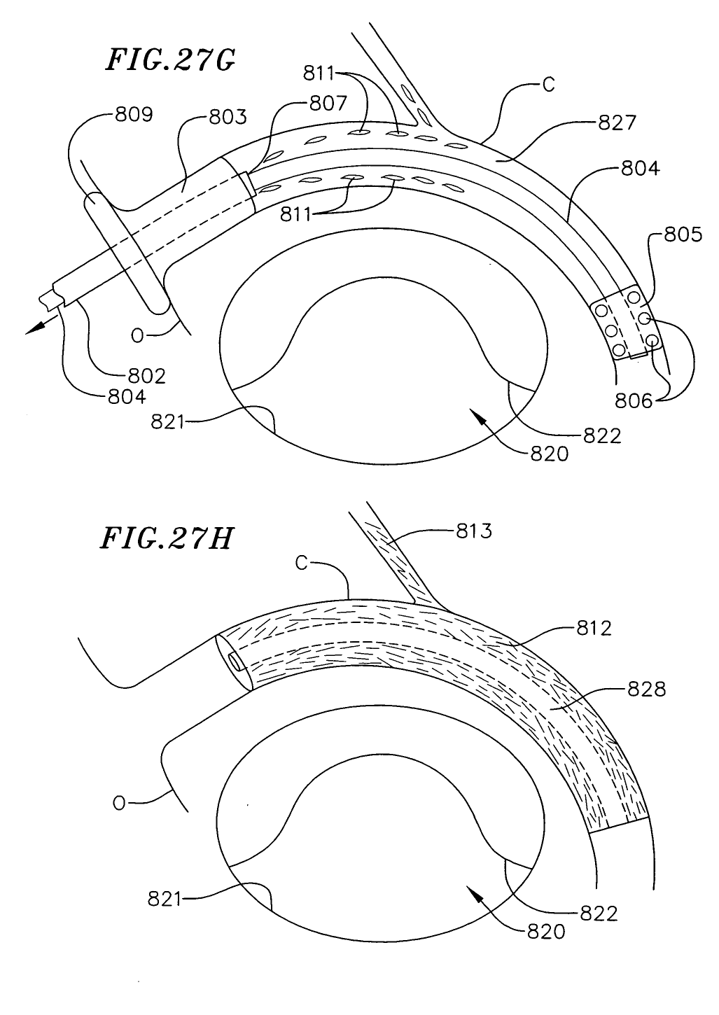

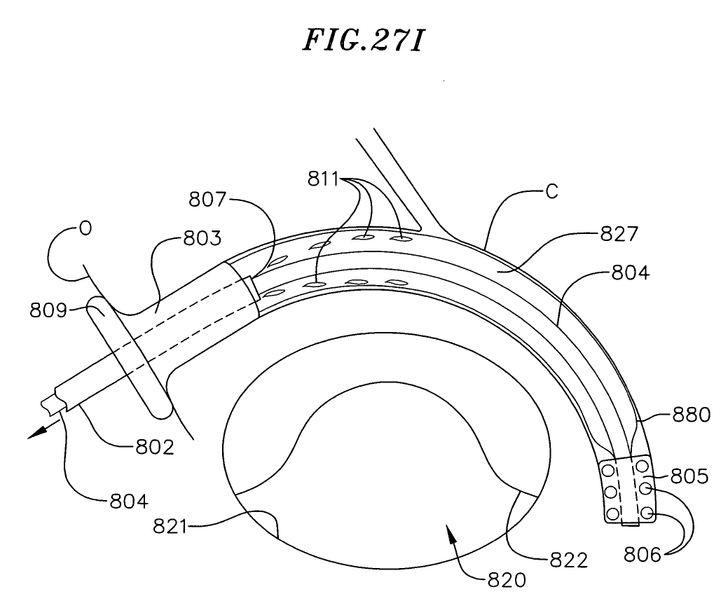

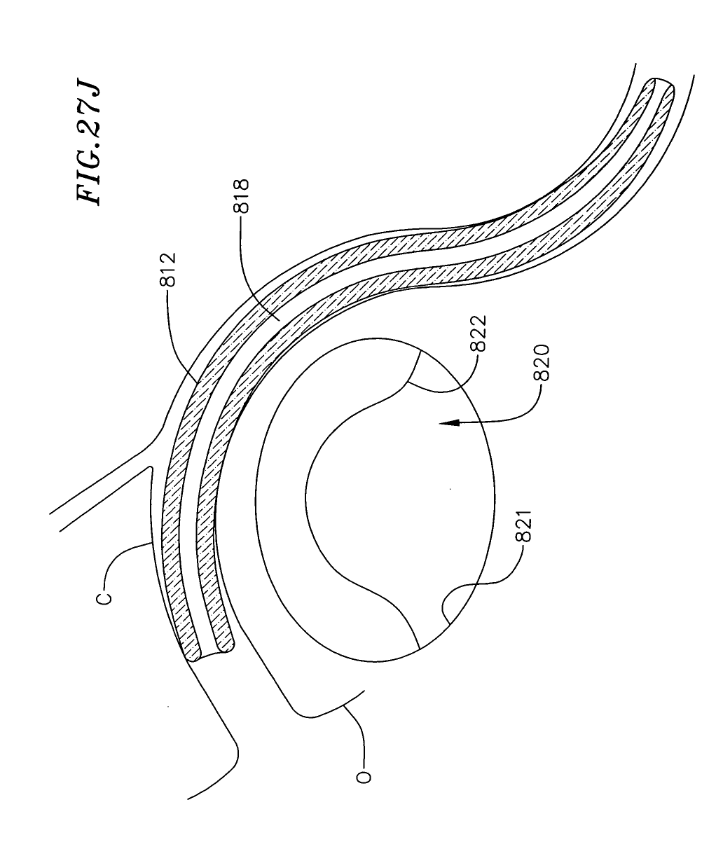

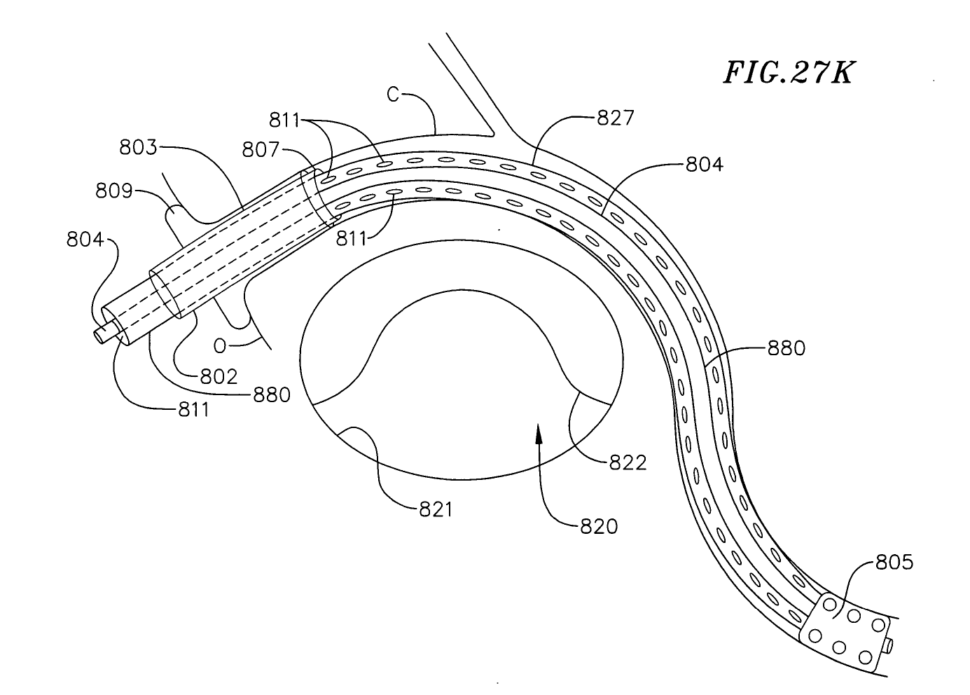

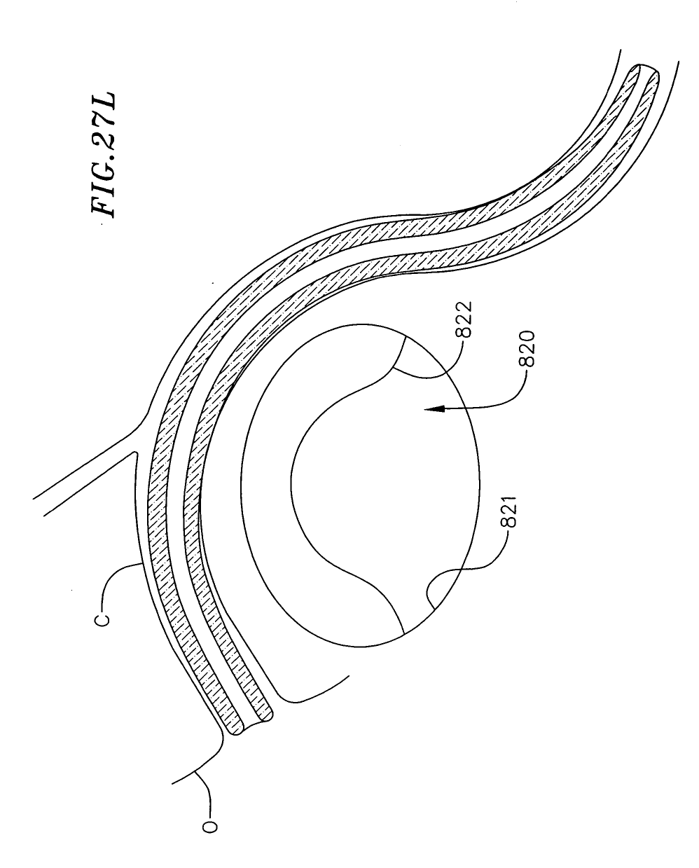

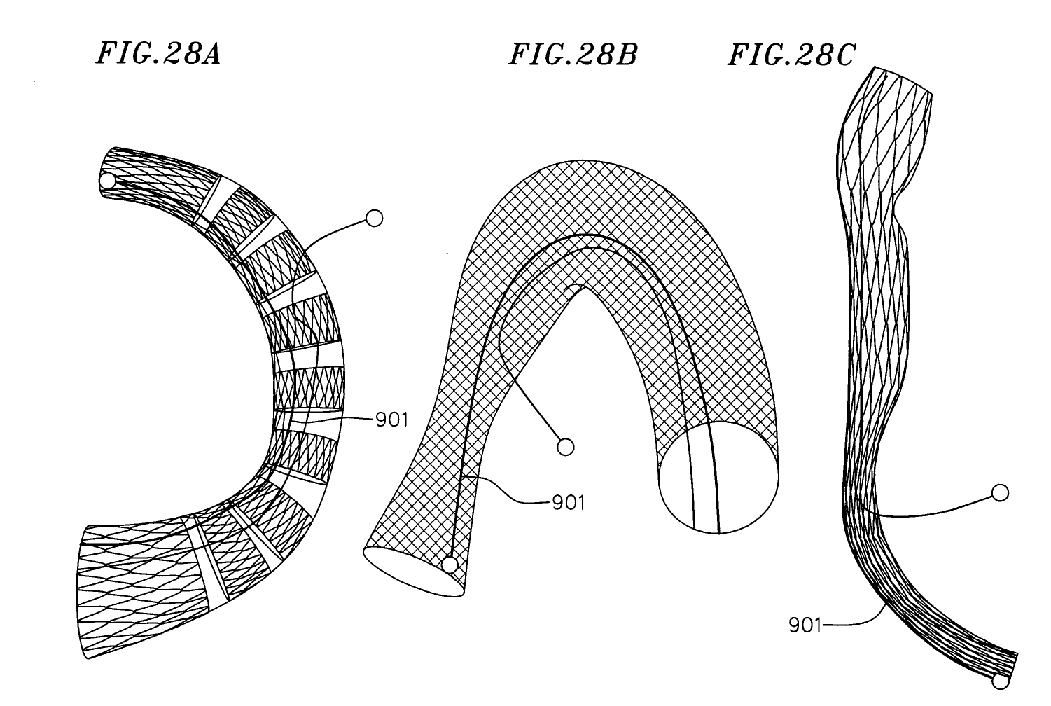

Referring now to FIGS. 27A-27L, alternative apparatus and methods suitable for treating mitral insufficiency are described. In FIG. 27A, distal balloon catheter 804 having proximal and distal ends, lumen 815 extending therebetween, and balloon 805 disposed at the distal end is positioned within coronary sinus C with balloon 805 in a contracted state. Distal catheter 804 may be positioned using a conventional guidewire (not shown), according to techniques that are known in the art. Distal catheter 804 further comprises an inflation lumen (not shown) extending between the proximal and distal ends that is in fluid communication with an opening of balloon 805, so that balloon 805 may be inflated via the inflation lumen, as shown in FIG. 27B.

Balloon 805 preferably includes a plurality of ribs or bumps 806 disposed about its circumference that are configured to engage the intima of a vessel wall and resist movement of balloon 805, when inflated, relative to the vessel.

After balloon 805 of distal catheter 804 is deployed in coronary sinus C, proximal balloon catheter 802 having proximal and distal ends, lumen 816 extending therebetween, and balloon 803 disposed at the distal end then may be advanced distally over distal catheter 804.

Lumen 816 of proximal catheter 802 comprises an inner diameter that is larger than an outer diameter of distal catheter 804, so that annulus 807 is defined as the space between an interior surface of proximal catheter 802 and an outer surface of distal catheter 804.

Proximal catheter 802 is provided with balloon 803 in a contracted state, and may be under fluoroscopy at a location whereby proximal section 819 of balloon 803 remains proximal of coronary ostium O, as shown in FIG. 27B. At this time, balloon 803 is inflated via an inflation lumen (not shown) of proximal catheter 802 to deploy balloon 803.

In the deployed state, balloon 803 of proximal catheter 802 comprises flange 809 disposed about proximal section 819 of balloon 803, as shown in FIG. 27C. In the deployed state, flange 809 is configured to abut against the wall of coronary ostium O, while a distal section of balloon 803 is configured to be substantially flush with the intima of coronary sinus C, as shown in FIG. 27C. An interior portion of coronary sinus C that is formed between deployed balloons 803 and 805 defines cavity 827.

Referring to FIG. 27D, balloon 805 of distal catheter 804 then may be retracted proximally and/or balloon 803 of proximal catheter 802 may be urged distally so that the distance between balloons 803 and 805 is reduced. Balloon 805 is disposed at such a distance apart from balloon 803 that the two balloons will apply a compressive force upon mitral valve 820 when the distance between balloons is reduced.

Ribs 806 of balloon 805 may engage the intima of coronary sinus C when balloon 805 is retracted, so that balloon 805 does not move with respect to coronary sinus C. Proximal retraction of balloon 805 causes coronary sinus C to shorten and remodel the curvature of the mitral valve annulus, as shown in FIG. 27D. The reduction in distance between balloons 803 and 805 applies a compressive force upon mitral valve 820 that reduces the circumference of mitral valve annulus 121 and thereby closes gap 822.

Referring now to FIG. 27E, with gap 822 reduced or closed as described hereinabove with respect to FIG. 27D, substance 811 then may be introduced into cavity 827 via annulus 807. Substance 811 may be a biological or synthetic biocompatible material that is injected in a fluid state, and which hardens to a rigid or semi-rigid state.

For example, substance 811 may comprise a biological hardening agent, such as fibrin, that induces blood captured in cavity 827 to form a coherent mass, or it may comprise a tissue material, such as collagen, that expands to fill the cavity. If fibrin is employed, it may be obtained from commercially available sources, or it may be separated out of a sample of the patient's blood prior to the procedure, and then injected into cavity 827 via annulus 807 to cause thrombosis. On the other hand, collagen-based products, such as are available from Collatec, Inc., Plainsboro, N.J., may be used to trigger thrombosis of the volume of blood in cavity 827.

Alternatively, substance 811 may comprise a synthetic molding material, such as a starch-based poly ethylene glycol hydrogel or a polymer, such as poly-caprolactone, that is heat hardenable or hydrophilic. In a preferred embodiment, a starch-based poly ethylene glycol hydrogel is used that swells when exposed to an aqueous solution. Hydrogels suitable for use with the present invention are described hereinabove with respect to FIG. 25B. Hydrogels or polymers also may be selected to harden, for example, upon exposure to body temperature or blood pH.

The injection of substance 811 between balloons 803 and 805 and into cavity 827 forms coherent mass 812, as shown in FIG. 27F. It is expected that, depending upon the type of hardening agent or molding material used, solidification of the content of cavity 827 may take about ten minutes or less.

After solidification of mass 812 has occurred, balloons 803 and 805 may be deflated. To facilitate removal of distal catheter 804 and balloon 805 from solidified mass 812, the exterior surface of distal catheter 804 and balloon 805 may be coated with a suitable non-stick coating, for example, Teflon®, a registered trademark of the E.I. duPont de Nemours Company, Wilmington, Del. (polytetrafluorethylene), or other suitable biocompatible material, such as Oparylene, available from Paratech®, Inc., Aliso Viejo, Calif. Proximal catheter 802 and/or balloon 803 also may be coated with such a non-stick coating to facilitate removal from within the patient's vessel.

Upon removal of proximal and distal catheters 802 and 804, solidified mass 812 maintains mitral valve 820 in the remodeled shape with gap 822 closed. The removal of distal catheter 804 from within solidified mass 812 may form bore 828 within the mass, as shown in FIG. 27F, which allows blood flow to be maintained within coronary sinus C. Because blood oxygenating the myocardium can drain directly into the left ventricle via the Thebesian veins, it is also permissible for the coronary sinus to be completely occluded with little or no adverse effect.

In an alternate embodiment of the present invention as shown in FIGS. 27G and 27H, the catheter 802 reaches all the way to the distal balloon 805. The distal balloon 805 with the catheter 802 is inserted into the great cardiac vein beyond where the vein turns away from the mitral valve plane at about 90 degrees. When a substance 811 is introduced into the device, the substance may also enter side branches 813 creating small arms there. These arms will aid in axially fixing the device once the substance is cured as described below. After the device has foreshortened as described above by moving the balloons 803, 805 towards each other and temporarily fixing their positions relative to each other, the lumen 816 of catheter 802 is filled with a substance 811 that when cured, for example by an ultraviolet light or by adding a proper chemical, becomes a hardened mass. Using this technique, a three-dimensional mass 812 having a small central bore 828 is created. This mass 812 is smaller in diameter than the coronary sinus C and the great cardiac vein, permitting close to normal blood flow in the vessel. Due to its three-dimensional shape and rigid configuration, the mass 812 is restricted to almost no axial movement. Thus, the shape of the coronary sinus C, the great cardiac vein and the mitral valve held temporarily by means of the two balloons 803, 805 may be held permanently by the mass 812.

In another embodiment as shown in FIGS. 27I and 27J, a film sack 880 is attached to the distal end of the proximal balloon 803. The diameter of the film sack is approximately equal to the diameter of the coronary sinus C and tapers down to approximately the diameter of the distal catheter 804 near the distal balloon 805 as shown in FIG. 27J. The film sack 880 is removably attached to the distal balloon 805 and may be manufactured from any thin plastic biocompatible material. A curable substance 811 is then introduced via the annulus 807 and cured by ultraviolet light or by the addition of a chemical as described above. When cured, the substance 811 forms a hardened mass that retains its shape and forces the affected vessels to also retain that shape. Once the substance 811 has hardened, the catheter 804, balloons 803, 805 and film sack 880 are removed.

In yet another embodiment, as shown in FIGS. 27K and 27L, the film sack 880 extends to outside the patient's body rather than being attached to the proximal balloon 803. Once the substance 811 is introduced, it can then be cured so as to form a hardened mass that extends all the way to the ostium O. This allows the cured substance to encompass a greater amount of the mitral valve annulus and ensures better closure of the gap created by mitral valve dilatation. The excess substance 811 that is not cured remains fluid and may be removed when the catheter 804, balloons 803, 805 and film sack 880 are removed.

Dilatation of the heart ventricles may lead to heart failure, which affects both the electrical and mechanical properties of the heart. Specifically, dilatation may cause distortion of the synchronization between the heart ventricles and atria. To correct this distortion, a pacemaker to stimulate contraction of the heart may be implanted into the heart, either through the chest wall or percutaneously through the venous system. Stent-type mechanisms are known that are connected to the tip of a pacing lead to securely anchor the pacing lead into a target vessel, such as those described in U.S. Pat. Nos. 5,071,407 (Termin, et al.), 5,224,491 (Mehra), 5,496,275 (Sirhan, et al.), 5,531,779 (Dahl, et al.) and 6,161,029 (Spreigl, et al.).

FIGS. 28A-28C illustrate another embodiment of the present invention. A pacing lead 901 such as described above may be attached to any of the previously described mitral valve annulus reshaping devices, for example elongate body 10 (FIG. 28A), elongate body 1300 (FIG. 28B) or elongate body 110 (FIG. 28C), to combine the function of the pacing lead with the function of the annulus reshaping device. Such a combination would allow for simultaneous treatment of arrhythmia and mitral regurgitation and would eliminate the need for a separate procedure to treat both conditions. Additionally, potential interference of the annulus reshaping device with the pacing lead would be avoided. As shown in FIGS. 28A-C, two pacing activity leads are used with each depicted elongate body which allows for effect at two locations. However, the number of pacing leads used is not critical and more or fewer than two leads may be used.

While the foregoing describes the preferred embodiments of the invention, various alternatives, modifications and equivalents may be used. For instance, although the described embodiments have generally been directed to placement in the coronary sinus for treatment of the mitral valve, the embodiments may also be placed in, for example, the anterior right ventricular cardiac vein to treat the tricuspid valve. Additionally, the order in which the stent sections of the various embodiments are expanded may be varied. Moreover, it will obvious that certain other modifications may be practiced within the scope of the appended claims.

1. A device for the treatment of mitral annulus dilatation comprising:
an elongate body having such dimensions as to be insertable into a coronary sinus, the elongate body including a distal stent section, a proximal stent section and a central stent section,
wherein the elongate body has two states,
a first state wherein the elongate body has a shape that is adaptable to the shape of the coronary sinus,
and a second state wherein the elongate body pushes on the coronary sinus to reduce dilatation,
wherein a backbone extends along the distal, proximal and central stent sections, the backbone includes alternating ring and severed regions fixing the stent sections relative to one another,
wherein the distal stent section and the proximal stent section in the second state anchor the device to the coronary sinus when the elongate body is positioned in the coronary sinus.

2. A device as in claim 1 wherein the central stent section has a plurality of alternating ring and severed regions.

3. A device as in claim 1 wherein the elongate body has a greater axial length in the first state than in the second state.

4. A device as in claim 1 wherein the elongate body includes a distal transitional section between the central stent section and the distal stent section and a proximal transitional section between the central stent section and the proximal stent section.

5. A device for treatment of mitral annulus dilation comprising:
an elongate, expandable body having a curved configuration to conform to an anatomy of a coronary sinus, the elongate, expandable body comprising a proximal stent section, a central stent section, and a distal stent section, wherein a diameter of the elongate, expandable body varies from the proximal stent section to the distal stent section,
the elongate, expandable body having two states,
a first state wherein the elongate, expandable body is compressed to be inserted into the coronary sinus; and
a second state wherein the elongate, expandable body self expands to correspond to a three dimensional shape of the coronary sinus;
the elongate body further having a third state shorter than the second state to provide foreshortening of the coronary sinus.

6. The device of claim 5, wherein the central stent section is located in an x-y plane and the distal stent section curves out of the x-y plane.

7. The device of claim 6, wherein the proximal stent section curves out of the x-y plane.

8. The device of claim 7, further comprising a sheath adapted to cover the elongate body when the elongate body is in the first state.

9. The device of claim 5, wherein the elongate body comprises a multi-filament woven structure.

10. The device of claim 5, wherein an end of the proximal stent section and an end of the distal stent section flare out in a funnel shape to provide greater anchoring capability.

11. The device of claim 5, further comprising a rigid inner elongate body adapted to be placed inside of the elongate body in the second state.

12. The device of claim 11, wherein the rigid inner elongate body is located within the central stent section of the elongate body.

13. A device for the treatment of mitral annulus dilatation comprising:
an elongate body having dimensions as to be insertable into a coronary sinus, the elongate body including
a first anchor;
a second anchor;
a first bridge between the first anchor and second anchor, wherein the bridge has open spaces and wherein the bridge has an elongated state in which the bridge has a first axial length and a shortened state in which the bridge has a second axial length, and
a resorbable thread to hold the bridge in the elongated state and to delay the transfer of the bridge to the shortened state when the device is inserted into the coronary sinus, the resorbable thread being woven between the open spaces of the bridge,
wherein the second axial length of the bridge is shorter than the first axial length,
wherein the bridge has a tendency to transfer its shape towards the shortened state when being in the elongated state.

14. The device of claim 13 wherein the resorbable thread is in compression.

15. The device of claim 13 wherein the bridge comprises X-shaped elements.

16. The device of claim 13 wherein the first anchor and the second anchor have two states, a compressed state and an expanded state.

17. The device of claim 16 wherein the first anchor has a greater diameter than the second anchor when both anchors are in the expanded state.

18. The device of claim 13 further comprising a third anchor and a second bridge between the second and third anchor, the second bridge having open spaces and an elongated state in which the second bridge has a first axial length and a shortened state in which the second bridge has a second axial length, and
a resorbable thread to hold the second bridge in the elongated state and to delay the transfer of the second bridge to the shortened state when the device is inserted into the coronary sinus, the resorbable thread being woven between the open spaces of the second bridge,
wherein the first axial length of the second bridge is shorter than the second axial length,
wherein the second bridge has a tendency to transfer its shape towards the shortened state when being in the elongated state.

19. The device as in claim 18 wherein the first bridge between the first anchor and the second anchor has a first length and the second bridge between the second anchor and the third anchor has a second length.

20. The device as in claim 19 wherein the length of the first bridge is different from the length of the second bridge.
